(19) United States
(12) Patent Application Publication
(10) Pub. No.: US 2024/0383968 A1
(43) Pub. Date: Nov. 21, 2024

(54) FORMULATION FOR MULTI-PURPOSE APPLICATION

(71) Applicant: BOEHRINGER INGELHEIM INTERNATIONAL GMBH, Ingelheim am Rhein (DE)

(72) Inventors: Michaela BLECH, Nonnenhorn (DE); Deborah BICKMANN, Gutenberg (DE); Sandra Nicole DENKINGER, Biberach an der Riss (DE); Silke MUEHLAU, Biberach an der Riss (DE)

(21) Appl. No.: 18/256,777

(22) PCT Filed: Dec. 10, 2021
(86) PCT No.: PCT/EP2021/085139

§ 371 Date: Jun. 9, 2023

(30) Foreign Application Priority Data
Dec. 11, 2020 (EP) 20213562.0
May 28, 2021 (WO) PCT/EP2021/064326
Nov. 24, 2021 (WO) PCT/EP2021/082892

(51) Int. Cl. C07K 16/10; A61K 9/00; A61K 9/08; A61K 47/12; A61K 47/18; A61K 47/26

(52) U.S. Cl. CPC C07K 16/1003; A61K 9/0019; A61K 9/0078; A61K 9/08; A61K 47/12; A61K 47/183; A61K 47/26

(57) ABSTRACT

The present invention relates in general to the formulation of antibodies and antigen-binding fragments thereof. In order to address a need for formulations for new antibodies that are able to be administered by different routes as well as in low and high dosage a formulation comprising an antibody or antigen-binding fragment thereof is provided, as well as uses of the formulation and methods involving the formulation.

### TECHNICAL FIELD

The present invention is in the field of formulations for biomolecules. It provides a formulation for antibody and antigen-binding fragments thereof, as well as uses thereof and methods involving the formulation.

### BRIEF SUMMARY OF THE INVENTION

The present invention relates in general to the formulation of antibodies and antigen-binding fragments thereof.

A formulation is provided comprising or consisting of an antibody or antigen-binding fragment thereof in an aqueous solution at a concentration of 1-200 mg/mL, 5-50 mM acetate or histidine, 120-260 mM glycine, 15-120 mM trehalose, 0.1-1.0 g/L polysorbate 20, and a pH of 4.5-6.5, as well as uses thereof and methods for treatment of a patient with the formulation.

### BRIEF DESCRIPTION OF THE DRAWINGS

FIG. 1: Stability data of mAb1 at 5° C. storage condition in Formulations F5 to F8, wherein the percentage of high molecular weight species (HMW (%)) is shown in A and the percentage of monomer (monomer (%)) is shown in B.

FIG. 2: Stability data of mAb1 at 25° C. storage condition in Formulations F5 to F8, wherein the percentage of high molecular weight species (HMW (%)) is shown in A and the percentage of monomer (monomer (%)) is shown in B.

FIG. 3: Stability data of mAb1 at 5° C. storage condition in Formulation F5 in different vial sizes, wherein the percentage of high molecular weight species (HMW (%)) is shown in A and the percentage of monomer (monomer (%)) is shown in B.

FIG. 4: Stability data of mAb1 at 25° C. storage condition in Formulation F5 in different vial sizes, wherein the percentage of high molecular weight species (HMW (%)) is shown in A and the percentage of monomer (monomer (%)) is shown in B.

FIG. 5: Stability data of mAb1 with different concentrations at 5° C. storage condition in Formulations F3 (10 mg/mL mAb1), to F4 (150 mg/mL mAb1), and F5 (50 mg/mL mAb1), wherein the percentage of high molecular weight species (HMW (%)) is shown in A and the percentage of monomer (monomer (%)) is shown in B.

FIG. 6: Stability data of mAb1 with different concentrations at 25° C. storage condition in Formulations F3 (10 mg/mL mAb1), to F4 (150 mg/mL mAb1), and F5 (50 mg/mL mAb1), wherein the percentage of high molecular weight species (HMW (%)) is shown in A and the percentage of monomer (monomer (%)) is shown in B.

FIG. 7: Load of SARS-COV-2 in nasopharyngeal swabs of animals pre-treated with mAb1 or vehicle prior to infection; LOD is limit of detection

FIG. 8: Load of SARS-COV-2 in bronchoalveolar lavages (BAL) of animals pre-treated with mAb1 or vehicle prior to infection; LOD is limit of detection

FIG. 9: Storage stability of mAb1 to mAb5 in formulation F5 at intended storage condition (5° C.), wherein the percentage of high molecular weight species (HMW (%)) is shown in A and the percentage of monomer (monomer (%)) is shown in B.

FIG. 10: Storage stability of mAb1 to mAb5 in formulation F5 at accelerated storage condition (25° C.), wherein the percentage of high molecular weight species (HMW (%)) is shown in A and the percentage of monomer (monomer (%)) is shown in B.

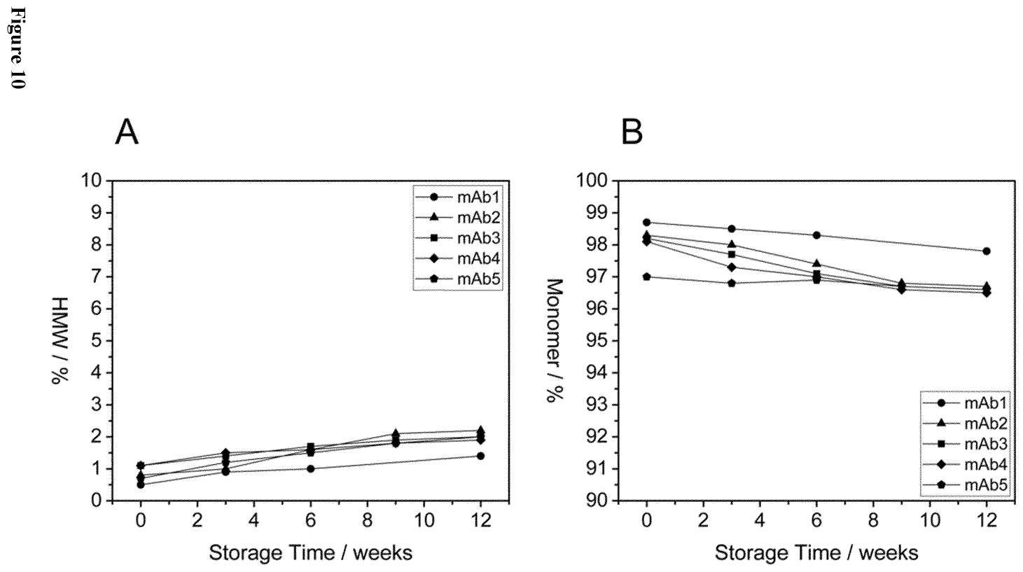

FIG. 11: Storage stability of mAb1 at 5° C. in formulations F1, F2, F5, and F6, wherein the percentage of high molecular weight species (HMW (%)) is shown in A and the percentage of monomer (monomer (%)) is shown in B.

FIG. 12: Storage stability of mAb2 at 5° C. in formulations F1, F2, F5, and F6, wherein the percentage of high molecular weight species (HMW (%)) is shown in A and the percentage of monomer (monomer (%)) is shown in B.

FIG. 13: Storage stability of mAb3 at 5° C. in formulations F1, F2, F5, and F6, wherein the percentage of high molecular weight species (HMW (%)) is shown in A and the percentage of monomer (monomer (%)) is shown in B.

FIG. 14: Storage stability of mAb4 at 5° C. in formulations F1, F2, F5, and F6, wherein the percentage of high molecular weight species (HMW (%)) is shown in A and the percentage of monomer (monomer (%)) is shown in B.

FIG. 15: Storage stability of mAb5 at 5° C. in formulations F1, F2, F5, and F6, wherein the percentage of high molecular weight species (HMW (%)) is shown in A and the percentage of monomer (monomer (%)) is shown in B.

FIG. 16: Storage stability of mAb1 at 25° C. in formulations F1, F2, F5, and F6, wherein the percentage of high molecular weight species (HMW (%)) is shown in A and the percentage of monomer (monomer (%)) is shown in B.

FIG. 17: Storage stability of mAb2 at 25° C. in formulations F1, F2, F5, and F6, wherein the percentage of high molecular weight species (HMW (%)) is shown in A and the percentage of monomer (monomer (%)) is shown in B.

FIG. 18: Storage stability of mAb3 at 25° C. in formulations F1, F2, F5, and F6, wherein the percentage of high molecular weight species (HMW (%)) is shown in A and the percentage of monomer (monomer (%)) is shown in B.

FIG. 19: Storage stability of mAb4 at 25° C. in formulations F1, F2, F5, and F6, wherein the percentage of high molecular weight species (HMW (%)) is shown in A and the percentage of monomer (monomer (%)) is shown in B.

FIG. 20: Storage stability of mAb5 at 25° C. in formulations F1, F2, F5, and F6, wherein the percentage of high molecular weight species (HMW (%)) is shown in A and the percentage of monomer (monomer (%)) is shown in B.

FIG. 21: Low concentration liquid formulation (LCLF) and high concentration liquid formulation (HCLF) storage stability data at intended storage condition (5° C.) of mAb1, mAb2, and mAb3, wherein the percentage of high molecular weight species (HMW (%)) is shown in A and the percentage of monomer (monomer (%)) is shown in B.

FIG. 22: Low concentration liquid formulation (LCLF) and high concentration liquid formulation (HCLF) storage stability data at accelerated storage condition (25° C.) of mAb1, mAb2 and mAb3, wherein the percentage of high molecular weight species (HMW (%)) is shown in A and the percentage of monomer (monomer (%)) is shown in B.

### BACKGROUND OF THE INVENTION

Antibodies and antigen-binding fragments of such antibodies have over the past decades gained importance in the field of pharmaceuticals. In particular in a pandemic situation, it has been shown that antibodies can be a source of specific treatment while other medications were still under evaluation.

One of the challenges when providing an antibody or antigen-binding fragment thereof as an active pharmaceutical ingredient (API), however, is the storage as well as the route of administration that is feasable considering the excipients that have to be present in a formulation. Most formulations of an antibody or an antigen-binding fragment thereof are specifically designed for one particular route of administration and/or one particular concentration of the API.

In pharmaceutical development, in particular in a pandemic situation, it is required to adapt quickly to a certain need in terms of providing formulations for new antibodies that are able to be administered by different routes as well as in low and high dosage.

### DETAILED DESCRIPTION OF THE INVENTION

In order to resolve the above identified need, a formulation was developed which has several advantages: Importantly, it represents a solution which can be used for multiple purposes such as for intravenous (i.v.) injection, subcutaneous (s.c.), and oral and nasal inhalation (inh.) administration. In addition, it can be used for pediatric use. In particular, it can be used both for an injection presentation as well as for a presentation for inhalation e.g. by means of a jet or mesh nebulizer.

Furthermore, the formulation as described is applicable especially for high dose administrations needed in pandemic situations or immunology, oncology (>1 g per patient per day) where commonly used excipients often exceed the level of maximum daily exposure limits for patients, and thus reaching critical toxicological level. In addition, sugar and polyols are commonly used to maintain the solution osmolality known to be essential for e.g. subcutaneous (s.c.) injection and cover the major proportion of commonly used formulations used in clinical/marketed products. However, for high dose administrations sugar or polyols often either exceed the maximum daily exposure levels for patients or fail to maintain solution osmolality.

The particular combination of excipients used in this formulation meet both the maximum daily exposure level for patients as well as for the solution osmolality evaluated for high dose administration of up to 5 g per patient per day even considering a 100 kg patient population.

Therefore, a pharmaceutical composition or formulation is generally provided herein comprising or consisting of an antibody or antigen-binding fragment thereof in an aqueous solution at a concentration of 1-200 mg/mL, 5-50 mM acetate or histidine, 120-260 mM glycine, 15-120 mM trehalose, 0.1-1.0 g/L polysorbate 20, and a pH of 4.5-6.5.

Surprisingly this formulation is capable of being administered to a patient via injection, including subcutaneous (s.c.), intravenous (i.v.), and intradermal, via inhalation, including oral, nasal, and combined (with a mask covering mouth and nose), without any adaptation of the formulation to the route of administration. The formulation can be readily used as low-concentrated liquid formulation (LCLF) and high-concentrated liquid formulation (HCLF) and stabilizes different antibodies over a wide range of concentrations and surface/volume ratios. Therefore, the formulation in addition provides administration both for low as well as high dosages in a patient.

Due to its' applicability over a wider range of concentrations of the API, its' capability to stabilize the API in that range, as well as its' availability for a number of different routes for administration to a patient, the formulation is suitable for multiple purposes.

In an embodiment, a formulation is provided, comprising or consisting of an antibody or antigen-binding fragment thereof in an aqueous solution at a concentration of 10-260 mg/mL, 10-25 mM acetate or histidine, 172.7-259.1 mM glycine, 17.3-25.9 mM trehalose, 0.2-0.6 g/L polysorbate 20 (polyoxyethylene (20)-sorbitan-monolaurate), and a pH of 5.2-5.8, The formulation provided has been shown to work for different antibodies.

In one embodiment, a pharmaceutical composition or formulation is provided comprising or consisting of an antibody in an aqueous solution at a concentration of 10-260 mg/mL, 10-25 mM acetate, 172.7-259.1 mM glycine, 17.3-25.9 mM trehalose, 0.2-0.6 g/L polysorbate 20 (polyoxyethylene (20)-sorbitan-monolaurate), and a pH of 5.2-5.8.

In another embodiment, a formulation is provided comprising or consisting of an antibody or antigen-binding fragment thereof in an aqueous solution at a concentration of 10 to 150 mg/mL, 20 mM acetate or histidine, 220 mM glycine, 20 mM trehalose, 0.4 g/L polysorbate 20 at a pH of 5.5.

In another embodiment, a pharmaceutical composition or formulation is provided comprising an antibody or antigen-binding fragment thereof at 50 mg/mL in 20 mM acetate, 220 mM glycine, 20 mM trehalose, 0.4 g/L polysorbate 20 at pH 5.5.

The formulation stabilizes all types of antibodies, including IgG1, IgG2, IgG3, IgG4, IgA, IgD, IgE, and IgM. In particular it is well suited in stabilizing antibodies of the type IgG1.

In one embodiment, the formulation has an osmolalilty of 220-380 mOsmol/kg, preferably 240-360 mOsmol/kg, more preferably 240-340 mOsmol/kg, even more preferably 260-320 mOsmol/kg.

Osmolality of the formulation is influenced among others by the nature, i.e. the molecular weight, and concentration of the antibody or antigen-binding fragment thereof.

The formulation was shown to be applicable as high-concentrated liquid formulation (HCLF), essential in order to accommodate high dose administration for s.c. injection of considerably low volume (max. 2.0 mL) into the patient using a syringe.

The formulation buffer, i.e. the aforementioned solution without active ingredient (antibody or antigen-binding fragment thereof) can be used as dedicated diluent, solvent for dilution, and placebo. It has further been shown to be compatible with commercial clinical dilution media.

In particular, an aqueous solution with 5-50 mM acetate or histidine, 120-260 mM glycine, 15-120 mM trehalose, 0.1-1.0 g/L polysorbate 20, and a pH of 4.5-6.5 can be used as dedicated diluent, solvent for dilution, placebo or diluent compatible to other clinical dilution media.

In another embodiment an aqueous solution with 10-25 mM acetate or histidine, 172.7-259.1 mM glycine, 17.3-25.9 mM trehalose, 0.2-0.6 g/L polysorbate 20 (polyoxyethylene (20)-sorbitan-monolaurate), and a pH of 5.2-5.8 is used as dedicated diluent, solvent for dilution, placebo or diluent compatible to other clinical dilution media.

In another embodiment an aqueous solution 20 mM acetate or histidine, 220 mM glycine, 20 mM trehalose, 0.4 g/L polysorbate 20 at a pH of 5.5 is used as dedicated diluent, solvent for dilution, placebo or diluent compatible to other clinical dilution media.

In yet another embodiment an aqueous solution with 20 mM acetate, 220 mM glycine, 20 mM trehalose, 0.4 g/L polysorbate 20 at pH 5.5 is used as dedicated diluent, solvent for dilution, placebo or diluent compatible to other clinical dilution media.

In an embodiment the formulation is diluted utilizing clinical dilution media, such as isotonic saline, Ringer lactate, or isotonic glucose solution.

In an embodiment the formulation comprises two or more antibodies or antigen-binding fragment thereof, preferably two.

In an embodiment the formulation is combined with another formulation comprising an antibody or antigen-binding fragment thereof.

In an embodiment the pharmaceutical formulation is administered at dosages of 10 to 50 mg/kg body weight, preferably 30 to 50 mg/kg body weight, of antibody or antigen-binding fragment thereof to a patient. The pharmaceutical formulation according to the invention is suitable for administration of up to 5 g antibody or antigen-binding fragment thereof to a patient per day, even when considering patients in a 100 kg body weight patient population, wherein at the same time the excipients do not exceed the maximum daily exposure level for patients while meeting the solution osmolality evaluated for high dose administration.

An antibody or an antigen-binding fragement thereof can be administered intravenously at doses of about 2.5 mg/kg, about 10 mg/kg, or about 40 mg/kg body weight by intravenous infusion diluted in formulation buffer. Thereby high dosage administration is in the range of 1 to 10 mg/kg body weight, whereas low dosage administration is in the range of 10 to 50 mg/kg body weight, preferaby 30 to 50 mg/kg body weight.

A pharmaceutical composition or formulation of the invention is formulated to be compatible with its intended route of administration. Examples of routes of administration include, but are not limited to, parenteral, e.g., intravenous, intradermal, subcutaneous, transdermal, but also intranasal, via inhalation (e.g., nasal inhalation and inhalation through the mouth), transmucosal, via rectal administration, among others.

In an embodiment the pharmaceutical formulation is used for administering an antibody or antigen-binding fragment thereof to a patient wherein the pharmaceutical formulation is administered by one or more of the following routes: injection, including intravenous, intradermal, and subcutaneous, inhalative, including oral inhalation, nasal inhalation, and mask inhalation, topical, including transdermal, transmucosal, and rectal, preferably the pharmaceutical formulation is administered intravenous, inhalative, including oral inhalation, nasal inhalation, and mask inhalation, and/or subcutaneous.

In a specific embodiment, the pharmaceutical composition is formulated in accordance with routine procedures as a pharmaceutical composition adapted for intravenous, subcutaneous, intramuscular, oral, intranasal, or topical administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection.

In an embodiment the formulation is nebulizable at a concentration of the antibody or antigen-binding fragment thereof of 1 to 150 mg/mL, preferably 10 to 80 mg/mL, more preferably 10 to 60 mg/mL, most preferably 10 to 50 mg/mL.

In an embodiment the pharmaceutical composition or formulation is administered by at least two different routes, the two different routes being selected from intravenous, inhalative, including oral inhalation, nasal inhalation, and mask inhalation, and/or subcutaneous.

In an embodiment the formulation is administered to a patient via at least two different routes and the concentration of the antibody or antigen-binding fragment thereof within the formulation that is administered by a first of the at least two different routes contains at least twice the concentration of the antibody or antigen-binding fragment thereof compared to the concentration of the antibody or antigen-binding fragment thereof within the formulation that is administered by the second of the at least two different routes.

In an embodiment of the invention, the pharmaceutical formulation is administered to a patient by inhalation, wherein the concentration of the antibody or antigen-binding fragment thereof in the formulation is from 1 to 150 mg/mL, preferably 10 to 80 mg/mL, more preferably 10 to 60 mg/mL, most preferably 10 to 50 mg/mL.

The formulation according to the invention can readily be administered by inhalation using different nebulizers, including mesh nebulizer, jet nebulizer, and ultrasonic nebulizer.

In an embodiment a method for treatment of a patient is provided wherein the pharmaceutical formulation is administered to the patient by one or more of the following routes: injection, including intravenous, intradermal, and subcutaneous, inhalative, including oral inhalation, nasal inhalation, and mask inhalation, topical, including transdermal, transmucosal, and rectal, preferably the pharmaceutical formulation is administered intravenous, inhalative, including oral inhalation, nasal inhalation, and mask inhalation, and/or subcutaneous.

In an embodiment a method for treatment of a patient is provided wherein the pharmaceutical formulation is administered by at least two different routes, the two different routes being selected from intravenous, inhalative, including oral inhalation, nasal inhalation, and mask inhalation, and/or subcutaneous.

A single inhalation may be followed by a single intravenous infusion.

In an embodiment a method for treatment of a patient is provided wherein an antibody or antigen-binding fragment thereof within the formulation is administered to the patient by at least two different routes and the concentration of the antibody or antigen-binding fragment thereof within the formulation that is administered by a first of the at least two different routes contains at least twice the concentration of the antibody or antigen-binding fragment thereof compared to the concentration of the antibody or antigen-binding fragment thereof within the formulation that is administered by the second of the at least two different routes.

In an embodiment of the invention a method for treatment of a patient is provided wherein the formulation is administered to the patient by inhalation and the concentration of the antibody or antigen-binding fragment thereof is from 10 to 150 mg/mL, preferably 10 to 80 mg/mL, more preferably 10 to 60 mg/mL, most preferably 10 to 50 mg/mL.

The pharmaceutical composition or the formulation as described could be demonstrated to efficiently stabilize mAb1 and other antibodies for inhalation administration using (i) different nebulizer systems (e.g. mesh nebulizer, jet nebulizer), (ii) diluted and undiluted formulation (different API concentration), and (iii) different masks or adapters for the inhaler (oral and nasal).

The methods of the invention may comprise pulmonary administration, e.g., by use of an inhaler or nebulizer, of a pharmaceutical composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In a specific embodiment, an antibody or antigen-binding fragment thereof, combination therapy, and/or pharmaceutical composition is administered using Alkermes AIR® pulmonary drug delivery technology (Alkermes, Inc., Cambridge, Mass., U.S.A.). In another specific embodiment, an antibody or antigen-binding fragment thereof, combination therapy, and/or pharmaceutical composition is administered using Aerogen Solo® pulmonary drug delivery technology (Aerogen GmbH, Ratingen, Germany).

For the inhaled administration, individuals may be treated with doses of e.g. 50 mg, 100 mg, or 250 mg per treatment through a mouthpiece following aerosol generation using a mesh nebulizer, or a jet nebulizer. The formulation may be diluted to the appropriate volume with formulation buffer.

The formulation was shown to stabilize the drug in different container closure systems, i.e. 20R (20 mL) and 6R (6 mL) Type vials, covering a wide range of technical parameter (e.g. surface/volume ratios).

The methods of the invention may also comprise administration of a pharmaceutical composition formulated for parenteral administration by injection (e.g. by bolus injection or continuous infusion), including subcutaneous, intravenous, intramuscular injection, among others.

The pharmaceutical formulation of the present invention may be provided in liquid form or may be provided in lyophilized form.

### Terms and Definitions

Terms not specifically defined herein should be given the meanings that would be given to them by one of skill in the art in light of the disclosure and the context. The general embodiments “comprising” or “comprised” encompass the more specific embodiment “consisting of”.

Furthermore, singular and plural forms are not used in a limiting way. As used herein, the singular forms “a”, “an”, “one” and “the” therefore refer to both the singular and the plural, unless otherwise indicated or apparent from the context.

As used in the specification, however, unless specified to the contrary, the following terms have the meaning indicated and the following conventions are adhered to. Terms used in the course of this present invention have the following meaning.

The terms “pharmaceutical composition” and “formulation” as used herein are meant to be interchangeable. The terms “pharmaceutical composition” and “formulation”, respectively, refer to a mixture of substances including a therapeutically active substance for pharmaceutical use, i.e. an active pharmaceutical ingredient (API). The term “formulation” or “pharmaceutical composition” in the context of this invention refers to a composition containing an antibody or antigen-binding fragment thereof, also referred to as active pharmaceutical or biological ingredient, along with one or more additional components.

The therapeutically active substance for pharmaceutical use herein is an antibody or antigen-binding fragment thereof.

“Antibodies and antigen-binding fragments thereof” are monoclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portions thereof retaining the binding specificities thereof, such as any specific binding portion thereof including those having any number of, immunoglobulin classes and/or isotypes (e.g., IgG1, IgG2, IgG3, IgG4, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region.

The term “antibody an antigen-binding fragment thereof” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab' fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody and antigen-binding fragment thereof” should be understood to encompass intact or full-length antibodies, including antibodies of any class or sub-class, including IgG and sub-classes thereof, IgM, IgE, IgA, and IgD, as well as function antibody fragments of the aforementioned. The antibody can comprise a human IgG1 constant region. The antibody can comprise a human IgG4 constant region.

The pharmaceutical composition or formulation according to the present invention may comprise a buffering agent. Buffering agents include, but are not limited to citric acid, HEPES, histidine, potassium acetate, potassium citrate, potassium phosphate (KH₂PO₄), sodium acetate, sodium bicarbonate, sodium citrate, sodium phosphate (NAH₂PO₄), Tris base, and Tris-HCl.

As used herein the term “buffering agent providing a pH of about 5.0 to about 7.0” refers to an agent which provides that the solution comprising it resists changes in pH by the action of its acid/base conjugate components. The buffer used in the formulations in accordance with the present invention may have a pH in the range from about 5.5 to about 7.5, or from about 5.8 to about 7.0. In one embodiment the pH is about 6.0. In one embodiment the pH is about 7.0. Examples of buffering agents that will control the pH in this range include acetate, succinate, gluconate, histidine, citrate, glycylglycine and other organic acid buffers.

The pharmaceutical composition or formulation according to the present invention may comprise a tonicity agent. Tonicity agents include, but are not limited to glycine, trehalose, dextrose, glycerin, mannitol, potassium chloride, and sodium chloride.

“Osmolality” means the concentration of a formulation in terms of osmoles of solutes per kilogram of solvent, i.e. water in an aqueous solution.

By “isotonic” is meant that the formulation has essentially the same osmotic pressure as human blood. Isotonic formulations will generally have an osmotic pressure from about 250 to 350 mOsmol/kg. Isotonicity can be measured using a vapor pressure or freezing-point depression type osmometer.

In certain embodiments, the pharmaceutical composition or formulation according to the present invention comprises a stabilizer. Stabilizers include, but are not limited to human serum albumin (hsa), bovine serum albumin (bsa), α-casein, globulins, α-lactalbumin, LDH, lysozyme, myoglobin, ovalbumin, and RNase A. Stabilizers also include amino acids and their metabolites, such as, glycine, alanine (α-alanine, β-alanine), arginine, betaine, leucine, lysine, glutamic acid, aspartic acid, proline, 4-hydroxyproline, sarcosine, γ-amino-butyric acid (GABA), opines (alanopine, octopine, strombine), and trimethylamine N-oxide (TMAO).

In certain embodiments, the pharmaceutical composition or formulation according to the present invention comprises a nonionic surfactant. Nonionic surfactants, include, but are not limited to, polyoxyethylensorbitan fatty acid esters (such as polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS). In one embodiment the nonionic surfactant is polysorbate 20 or polysorbate 80. In one embodiment the polysorbate concentration is about 0.005 to 0.02% (w/v).

The term “nebulizable” refers to the ability of the formulation to form soluble aerosol droplets characterized as a nebulizer solution according to the EMA/CHMP/QWP/49313 “Guideline on the pharmaceutical quality of inhalation and nasal products” in combination with Ph. Eur. 2.9.44 and the FDA Guidance for Industry “Nasal Spray and Inhalation Solution, Suspension, and Spray Drug Products—Chemistry Manufacturing, and Controls Documentation” in combination with USP <1601>. The content of each of these documents is herewith incorporated by reference in their entireties.

For determining whether a formulation is “nebulizable” in the sense used herein, accordingly, the following parameter describing the aerodynamic performance are investigated:
Active Substance Delivery Rate (ASDR)/Drug Substance Delivery Rate (DSDR)
Total Active Substance Delivered (TASD)/Total Drug Substance Delivered (TDSD)
Aerodynamic Assessment of Nebulized Aerosols:
   particle size distribution,
   mass median aerodynamic diameter (MMAD)
   geometric standard deviation (GSD)
   fine particle fraction (FPF)<5.0 μm

Devices for investigating whether a formulation is “nebulizable” are CE marked mesh or jet nebulizers and have 510 k clearance. These are intended to deliver nebulization solutions. For the devices used herein suitability for the delivery of solutions for nebulization was confirmed in Aerogen® Solo System instruction manual, R20-3604, InnoSpire® Go instruction manual, R20-4198, and Aero Eclipse * II Breath Actuated Nebulizer (BAN) Instruction Manual, R20-4199. These devices should be used for comparability of results.

For assessment of the complete delivery profile of the product for use in clinical trials, the ASDR/DSDR and TASD/TDSD (i.e. total dose delivered to the patient) is determined according to the procedure described in Ph. Eur. 2.9.44 and in USP <1601> with the exception that individual experiments are conducted for the measurement of these two parameters. ASDR/DSDR and TASD/TDSD are investigated using a filter-based method including an artificial lung as breathing simulator (ASL 5000, Ingmar Medical, Pittsburgh, PA, USA; 500 mL inhaled volume, tidal breathing, 15 breaths per minute, sinusoidal, 1:1 inhalation/exhalation ratio).

The particle size distribution of liquid aerosol droplets generated by the nebulizers is assessed using a next generation impactor (NGI). Mass median aerodynamic diameter (MMAD), geometric standard deviation (GSD) and fine particle fraction (FPF<5.0 μm) are calculated from the impactor measurements.

The Mass Median Aerodynamic Diameter (MMAD) aerodynamic diameter of a droplet or particle is equal to the diameter of a sphere with a density of 1 g/mL which has the same settling velocity. Besides the geometric diameter, the density and shape of the particle influences the aerodynamic properties. In general, MMAD values between 1 to 5 μm are considered optimal for lung deposition. Smaller particles are exhaled, while larger particles are deposited in mouth, nose, or trachea.

The Geometric Standard Deviation (GSD) is a measure of dispersion for the deviation from the mean value of the size distribution, the MMAD. It is a dimensionless number. Small values indicate a narrow size distribution, which is preferred for pulmonary application, as more droplets or particles should be in the desired range between 1 to 5 μm, if the MMAD is in this region.

The Fine Particle Fraction (FPF) is the proportion of particles or droplets in the aerosol with a diameter below 5.0 μm. This is the portion that reaches the deeper lungs after inhalation.

Ultra-performance size-exclusion chromatography (UP-SEC) is used to determine high molecular weight (HMW) and monomer content prior and after nebulization in order to evaluate the antibody integrity.

Surface plasmon resonance (SPR) is used to assess the binding activity, and thus for evaluation whether the antibodies retain function after nebulization. UV-Vis, UP-SEC, and SPR is used to assess the antibody's integrity, function, and product quality.

“Nasal inhalation” means inhaling through only the nose using, e.g. a nasal cast whereby the nose is completely covered and aerosol droplets enter the respiratory tract via the nose.

“Oral inhalation” means inhaling through only the mouth using, e.g. a mouth piece whereby the liquid aerosol droplets enter the respiratory tract via the mouth/throat.

Mask inhalation means inhalation through the nose and mouth simultaneously whereby the facial mask covers both the nose and the mouth. The uptake of liquid aerosol droplets is delivered through both the nose and mouth/throat to the respiratory tract.

### EXAMPLES

Different monoclonal antibodies have been used in order to show the effects of the formulation.

Antibody 1 (mAb1) is a monoclonal antibody described as DZIF-10c in patent applications EP20213562.0 and PCT/EP2021064326. This antibody is of the type IgG 1.

mAb2 is a monoclonal antibody of the type IgG4. The antibodies mAb3 to mAb5 are monoclonal antibodies, as well, all of the type IgG1.

### Example 1.1

Formulation of mAb1 and Other Antibodies

mAb1 can be provided as a single-use sterile solution at a concentration of 50 mg/mL. Each vial of mAb1 drug product may contain 20 mL of a buffered solution composed of acetic acid, sodium acetate, glycine, trehalose, and polysorbate 20 (see table 1).

TABLE 1

| Component | Concentration[mg/mL] |
|---|---|
| mAb1 | 50.00 |
| Acetic acid glacial | 0.25 |
| Sodium acetate trihydrate | 2.15 |
| Glycine | 16.52 |
| Trehalose dihydrate | 7.57 |
| Polysorbate 20 | 0.40 |
| Water for Injection (WFI) | Ad 1 mL |

In an embodiment, mAb1 is formulated at about 50 mg/mL in about 20 mM acetate, about 220 mM glycine, about 20 mM trehalose, about 0.4 g/L polysorbate 20 at a pH of about 5.5.

mAb1 can be applied by intravenous infusion or inhalation administration after aerosolization using a nebulizer.

mAb1 has been administered intravenously to patients in a clinical study at doses of about 2.5 mg/kg, about 10 mg/kg, or about 40 mg/kg by intravenous infusion diluted in formulation buffer over 60 minutes (+/−10 minutes) using a 0.2 μm nylon in-line filter (results not shown here). The formulation exemplified above may be diluted to the appropriate volume with formulation buffer, i.e. the above formulation without the antibody mAb1.

For the inhaled administration, individuals may be treated with doses of e.g. 50 mg, 100 mg, or 250 mg per treatment through a mouthpiece following aerosol generation using a mesh nebulizer, or a jet nebulizer. The formulation exemplified above may be diluted to the appropriate volume with formulation buffer.

A single inhalation may be followed by a single intravenous infusion.

In below table 2 different formulations for antibodies are provided, which are partially according to the invention and partially comparative formulations. Formulation F5 corresponds to the formulation in table 1.

TABLE 2
Example formulations

| Annotation | Buffer | pH | Excipient 1 | Excipient 2 | PS20 | API conc. | |
|---|---|---|---|---|---|---|---|
| F1 | 20 mM Phosphate | 7.5 | — | 240 mM Trehalose | 0.4 g/L | 50 | mg/mL |
| F2 | 20 mM Acetate | 5.5 | 150 mM NaCl | — | 0.4 g/L | 50 | mg/mL |
| F3 | 20 mM Acetate | 5.5 | 220 mM Glycine | 20 mM Trehalose | 0.4 g/L | 10 | mg/mL |
| F4 | 20 mM Acetate | 5.5 | 220 mM Glycine | 20 mM Trehalose | 0.4 g/L | 150 | mg/mL |
| F5 | 20 mM Acetate | 5.5 | 220 mM Glycine | 20 mM Trehalose | 0.4 g/L | 50 | mg/mL |
| F6 | 20 mM Citrate | 6.5 | 220 mM Glycine | 20 mM Trehalose | 0.4 g/L | 50 | mg/mL |
| F7 | 20 mM Histidine | 6.0 | 220 mM Glycine | 20 mM Trehalose | 0.4 g/L | 50 | mg/mL |
| F8 | 20 mM Histidine | 6.0 | — | 180 mM Trehalose | 0.4 g/L | 50 | mg/mL |
| F9 | 20 mM Acetate | 5.5 | 220 mM Glycine | 20 mM Trehalose | 0.4 g/L | 100 | mg/mL |

API means “active pharmaceutical ingredient” and reflects the amount of antibody added.

Formulations F3 to F5, and F9 are according to the invention, including different amounts of antibody. Formulation F7 is according to the invention, as well, comprising histidine instead of acetate. All other formulations are comparative examples. The comparative examples are commonly used for commercially available antibodies, e.g. formulation F1, F2, and F8.

### Example 1.2

Comparison of Different Formulations of mAb1 in Terms of Stability

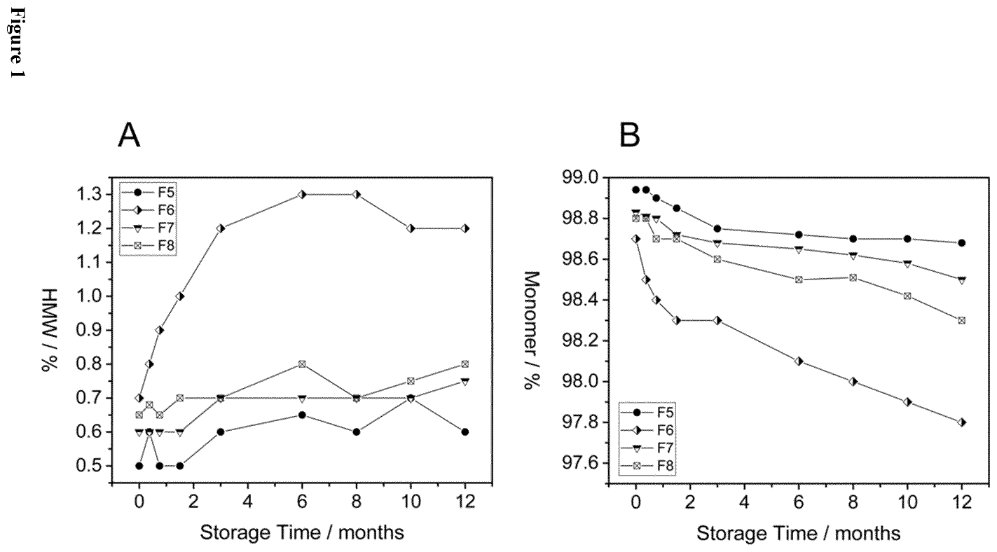

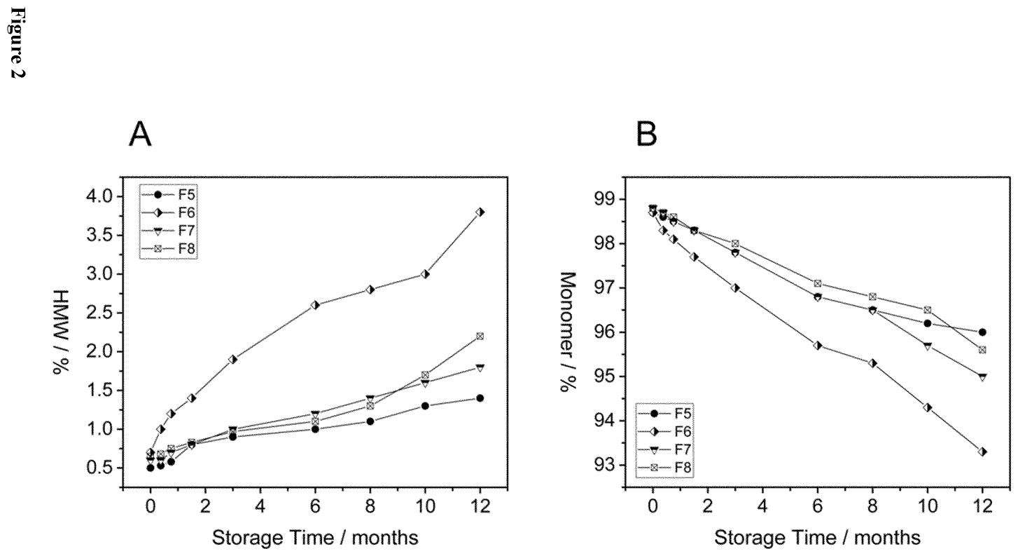

Stability data of formulations F5 to F8 with mAb1 at intended (5° C.) storage condition and accelerated storage conditions (25° C.) have been measured via the percentage of high molecular weight species (HMW (%)) or via the percentage of monomer (monomer (%)) over a storage time of up to 12 months. Results of these measurements are shown in FIGS. 1 and 2 for storage conditions at 5° C. and 25° C., respectively, wherein the percentage of HMW is shown in A and the percentage of monomer is shown in B. The Monomer represents the antibody's active form.

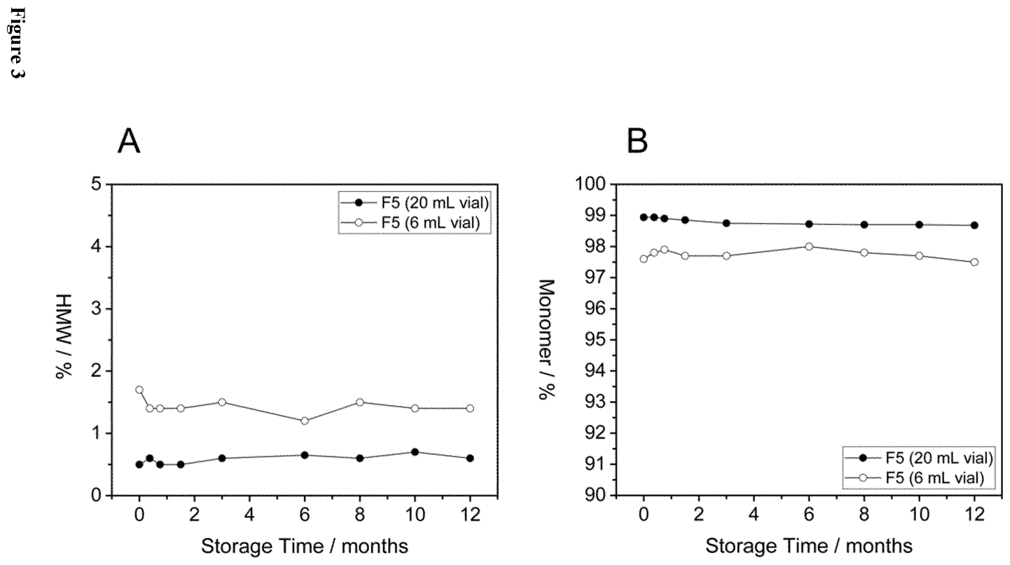

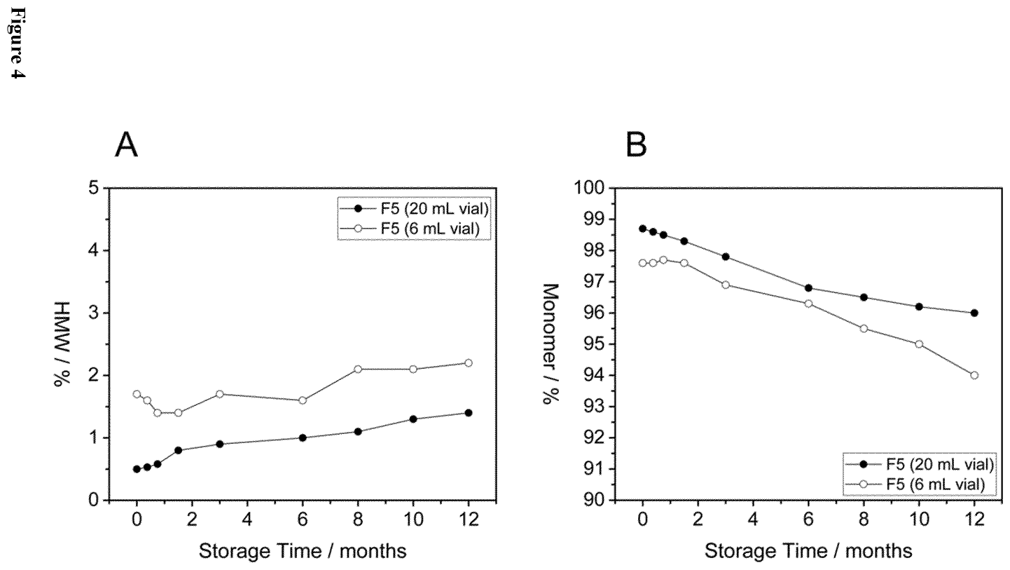

Formulation F5 with mAb1 was shown to be stable in different container closure systems, namely 20R (20 mL) and 6R (6 mL), covering a wide range of technical parameter (e.g. surface/volume ratios). For the experiments Type I glass vials have been used. This data is shown in FIGS. 3 and 4, representing storage conditions at 5° C. and 25° C., respectively, wherein the percentage of HMW is shown in A and the percentage of monomer is shown in B.

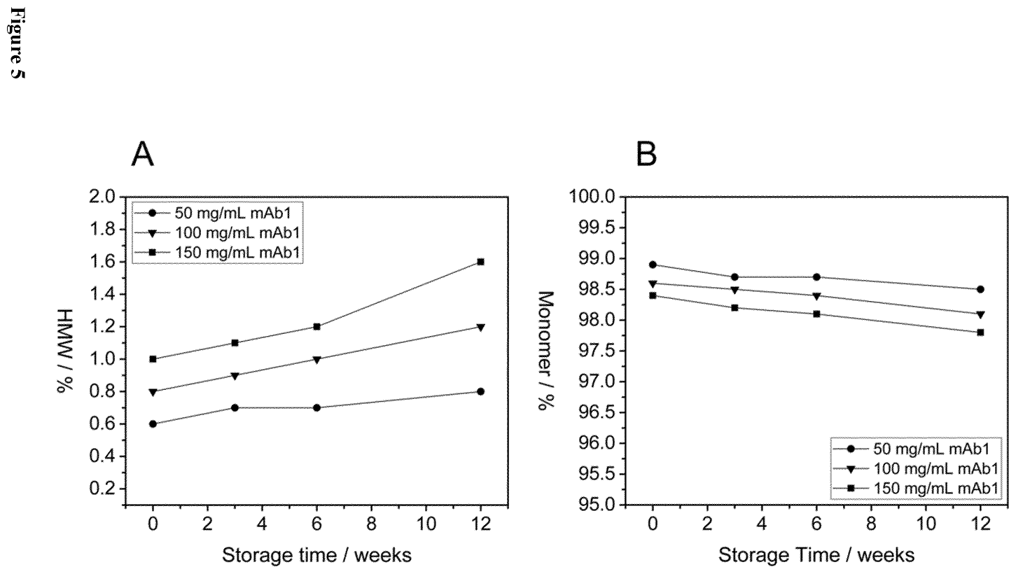

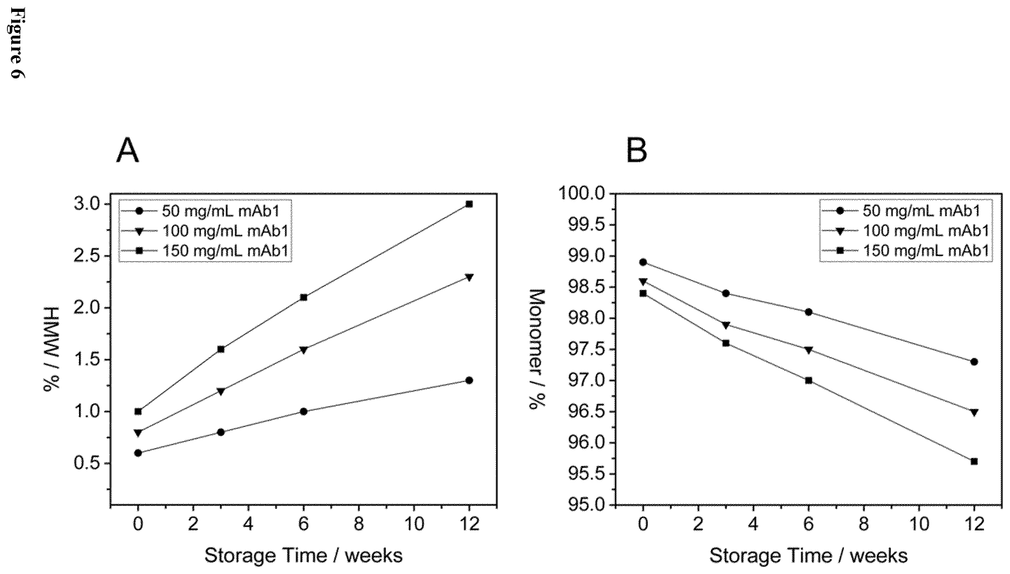

Further data in FIGS. 5 and 6 show a good stability of mAb1 in the formulations according to the invention over a wide range of concentrations of the antibody, namely in formulations F4, F5, and F9, wherein F9 has a concentration of 100 mg/ml mAb1, F4 has a concentration of 150 mg/mL mAb1, both representing a high-concentrated liquid formulation (HCLF), and F5 has a concentration of 50 mg/mL mAb1. Data have been obtained over twelve weeks of storage time. In each of the figures the percentage of HMW is shown in A and the percentage of monomer is shown in B.

### Example 1.3

mAb1 is Nebulizable in Formulation F5

The nebulization characteristics of mAb1 in F5 were assessed with the mesh nebulizer Aerogen® Solo. The respective MMAD, GSD and FPF <5.0 μm, were determined using an NGI. The size limit of 5.0 μm particle diameter is stipulated in Ph. Eur. 2.9.18; it is generally accepted that particles with an aerodynamic diameter smaller than 5.0 μm can be assumed to reach the lower respiratory tract (deeper lung region).

TABLE 3
Aerodynamic assessment of mAb1 at 50 mg/mL in F5 with the Aerogen ® Solo
investigating three independent prepared batches

| | mAb1 batch1 | | | mAb1 batch 2 | | | mAb1 batch 3 | | |
|---|---|---|---|---|---|---|---|---|---|
| | | | FPF | | | FPF | | | FPF |
| | MMAD | | <5 μm | MMAD | | <5 μm | MMAD | | <5 μm |
| Assessment | [μm] | GSD | [%] | [μm] | GSD | [%] | [μm] | GSD | [%] |
| Test 1 | 4.45 | 2.1 | 57 | 4.14 | 2.1 | 61 | 4.31 | 2.1 | 58 |
| (n = 3) | | | | | | | | | |
| Test 2 | 4.34 | 2.2 | 58 | 3.72 | 2.3 | 66 | 4.32 | 2.1 | 58 |
| (n = 3) | | | | | | | | | |
| Test 3 | 4.50 | 2.0 | 56 | 4.46 | 2.1 | 57 | 4.30 | 2.2 | 59 |
| (n = 3) | | | | | | | | | |
| Mean | 4.43 | 2.1 | 57 | 4.11 | 2.2 | 61 | 4.31 | 2.1 | 58 |

The particle size distribution of liquid aerosol droplets generated by the nebulizers was assessed using a next generation impactor (NGI). Therefore, the NGI was pre-cooled at about 5° C. before use and measurements were started within 5 min after removal from the refrigerator. The impactor was operated at a flow rate of 15 L/min. A fill volume of 1 mL was used for the mesh nebulizers and nebulization was continued until end of aerosol generation.

Mass median aerodynamic diameter (MMAD), geometric standard deviation (GSD) and fine particle fraction (FPF<5.0 μm) were calculated from the impactor measurements.

Table 3 provides the calculated values of MMAD, GSD and FPF<5.0 μm of liquid aerosol droplets generated by the Aerogen® Solo. The data in table 3 provide information about the suitability of F5 for inhalation in general. In addition, it shows the variability between measurements performed of three independently prepared mAb1 formulated in F5 batches in combination with the respective Aerogen® Solo nebulizer. A test in this setting corresponds to one individual Aerogen® Solo nebulizer whereas the nebulization experiment for the individual mAb1 batches were performed in triplicates. In summary, three independently prepared mAb batches were each nebulized in each of the three different Aerogen® nebulizers.

Overall, all three parametes are comparable for the tested batches and nebulizers showing consitency and robustness of the nebulization for mAb in F5 with respect to the aerodynamic assessment.

The respective FPF<5.0 μm for all mAb1 batches are close to 60% which means that approximately 60% of mAb1 enter the lower respiratory tract (deeper lung region) and accounts for good nebulization performance among the other obtained aerodynamic parameter.

In summary, F5 nebulizer solution is suitable for adequate inhalation administration to patients using the Aerogen® Solo system (see table 3).

### Example 1.4

Comparison of Different Nebulizers for Nebulization of mAb1

For all assessments, mAb1 formulated in F5, three independent replicate tests were performed with one nebulizer per tested batch. Different nebulizers were used for the individual batches.

Formulation F5 showed stabilizing effect when tested in different commercially available nebulizer systems (mesh and jet nebulizers), see table 4.

TABLE 4
Product quality parameters upon nebulization of mAb1 formulated
in F5 using different mesh and jet nebulizer

| | | | | | Phillips | | |
|---|---|---|---|---|---|---|---|
| | | | Aerogen | eFlow- | Inno- | Pari- | |
| | | Control | Solo ® | Rapid ® | SpireGo ® | boy ® | Droh ® |
| | | unnebulized | nebulized | nebulized | nebulized | nebulized | nebulized |
| | | — | mesh | mesh | mesh | jet | jet |
| UV/Vis | | 48.6 | 48.3 | 48.8 | 48.8 | 61.3 | 68.8 |
| (mg/mL) | | | | | | | |
| Osmolality | | 291 | 292 | 291 | 293 | 374 | 429 |
| (mOsmol/kg) | | | | | | | |
| Surface | | 38.2 | N/A | N/A | N/A | N/A | N/A |
| Tension | | | | | | | |
| (mN/m) | | | | | | | |
| UP-SEC | HMW | 0.6 | 0.5 | 0.5 | 0.5 | 0.5 | 0.5 |
| (%) | Monomer | 98.8 | 98.9 | 98.9 | 98.9 | 98.9 | 98.9 |
| | LMW | 0.6 | 0.6 | 0.6 | 0.6 | 0.6 | 0.6 |
| CGE | HMW | 0.2 | 0.2 | 0.2 | 0.2 | 0.2 | 0.2 |
| (non-red) | Monomer | 96.5 | 96.6 | 96.4 | 96.5 | 96.5 | 96.4 |
| (%) | LMW | 3.4 | 3.3 | 3.5 | 3.3 | 3.4 | 3.4 |
| retained | | 100 | 100 | 100 | 100 | 100 | 100 |
| binding | | | | | | | |
| Activity* | | | | | | | |
| (%) | | | | | | | |

N/A corresponds to not analyzed
*measurements performed using surface plasmon resonance (SPR)

Osmolality was determined after nebulization. When using a jet nebulizer a higher osmolality was observed.

In summary, it was shown that integrity of mAb1 was not affected by nebulization with the different nebulizer devices.

### 1.5—Comparison of Different Formulations of mAb1 in Terms of Nebulization

Different formulations according to Table 2 were tested in the Aerogen Solo® nebulizer, i.e. in a mesh nebulizer (table 5). The same measurement was done with an API concentration of 10 mg/mL (table 6). In order to obtain the lower concentration of 10 mg/mL of mAb1, the formulations F5, F6, F7, and F8 have been diluted with formulation buffer, whereby for each sample the same composition as for the formulation has been used as a diluent, however, without API.

TABLE 5
Results of undiluted formulations (50 mg/mL mAb1)

| | | F5 | | F6 | | F7 | | F8 | |
|---|---|---|---|---|---|---|---|---|---|
| | | initial | nebulized | initial | nebulized | initial | nebulized | initial | nebulized |
| UV/Vis | | 50.4 | N/A | 50.2 | N/A | 50.9 | N/A | 50.8 | N/A |
| (mg/mL) | | | | | | | | | |
| pH | | 5.5 | 5.5 | 6.4 | 6.4 | 6.0 | 6.0 | 6.0 | 6.0 |
| (—) | | | | | | | | | |
| Osmolality | | 292 | N/A | 322 | N/A | 284 | N/A | 241 | N/A |
| (mOs- | | | | | | | | | |
| mol/kg) | | | | | | | | | |
| Surface | | 38.98 | N/A | 39.93 | N/A | 39.00 | N/A | 38.56 | N/A |
| Tension | | | | | | | | | |
| (mN/m) | | | | | | | | | |
| Viscosity | | 1.76 | N/A | 1.85 | N/A | 1.82 | N/A | 1.97 | N/A |
| at 20° C. | | | | | | | | | |
| (mPas) | | | | | | | | | |
| Density | | 1.023 | N/A | 1.026 | N/A | 1.024 | N/A | 1.038 | N/A |
| at 20° C. | | | | | | | | | |
| (g/mL) | | | | | | | | | |
| UP-SEC | HMW | 0.4 | 0.4 | 0.7 | 0.7 | 0.4 | 0.4 | 0.4 | 0.4 |
| (%) | Monomer | 99.1 | 99.0 | 98.7 | 98.7 | 99.0 | 99.0 | 99.0 | 99.0 |
| | LMW | 0.6 | 0.6 | 0.6 | 0.6 | 0.6 | 0.6 | 0.6 | 0.6 |
| CGE | HMW | 0.1 | 0.1 | 0.3 | 0.3 | 0.2 | 0.1 | 0.2 | 0.2 |
| non- | Monomer | 96.6 | 96.6 | 96.4 | 96.5 | 96.5 | 96.5 | 96.5 | 96.5 |
| reduced | LMW | 3.3 | 3.3 | 3.2 | 3.3 | 3.3 | 3.3 | 3.3 | 3.4 |
| (%) | | | | | | | | | |
| CGE | HMW | 1.1 | 1.1 | 1.2 | 1.1 | 1.0 | 1.0 | 1.0 | 1.0 |
| reduced | HC + LC | 97.9 | 98.0 | 97.8 | 98.0 | 98.1 | 98.1 | 98.0 | 98.0 |
| (%) | LMW | 1.0 | 1.0 | 1.0 | 0.9 | 0.9 | 0.9 | 1.0 | 1.0 |
| retained | | 100 | 100 | 100 | 100 | 100 | 100 | 100 | 100 |
| binding | | | | | | | | | |
| Activity * | | | | | | | | | |
| (%) | | | | | | | | | |

N/A corresponds to not analyzed
* measurements performed using surface plasmon resonance (SPR)

TABLE 6
Results of 1:5 diluted formulations (10 mg/mL mAb1)

| | | F5# | | F6# | | F7# | | F8# | |
|---|---|---|---|---|---|---|---|---|---|
| | | initial | nebulized | initial | nebulized | initial | nebulized | initial | nebulized |
| UV/Vis | | 9.9 | N/A | 9.9 | N/A | 9.9 | N/A | 10.1 | N/A |
| (mg/mL) | | | | | | | | | |
| pH | | 5.5 | 5.5 | 6.5 | 6.5 | 6.0 | 6.0 | 6.0 | 6.0 |
| (—) | | | | | | | | | |
| Osmolality | | N/A | N/A | N/A | N/A | N/A | N/A | N/A | N/A |
| (mOs- | | | | | | | | | |
| mol/kg) | | | | | | | | | |
| Surface | | 39.29 | N/A | 40.20 | N/A | 40.08 | N/A | 38.56 | N/A |
| Tension | | | | | | | | | |
| (mN/m) | | | | | | | | | |
| Viscosity | | 1.21 | N/A | 1.23 | N/A | 1.23 | N/A | 1.40 | N/A |
| at 20° C. | | | | | | | | | |
| (mPas) | | | | | | | | | |
| Density | | 1.012 | N/A | 1.015 | N/A | 1.012 | N/A | 1.027 | N/A |
| at 20° C. | | | | | | | | | |
| (g/mL) | | | | | | | | | |
| UP-SEC | HMW | 0.4 | 0.4 | 0.7 | 0.7 | 0.4 | 0.5 | 0.4 | 0.5 |
| (%) | Monomer | 99.1 | 99.0 | 98.7 | 98.6 | 99.0 | 99.0 | 99.0 | 99.0 |
| | LMW | 0.6 | 0.6 | 0.6 | 0.6 | 0.6 | 0.6 | 0.6 | 0.6 |
| CGE | HMW | 0.1 | 0.1 | 0.3 | 0.4 | 0.2 | 0.2 | 0.2 | 0.2 |
| non- | Monomer | 96.6 | 96.6 | 96.4 | 96.2 | 96.4 | 96.4 | 96.3 | 96.2 |
| reduced | LMW | 3.3 | 3.3 | 3.2 | 3.4 | 3.4 | 3.5 | 3.5 | 3.5 |
| (%) | | | | | | | | | |
| CGE | HMW | 1.1 | 1.1 | 1.2 | 1.3 | 0.9 | 1.0 | 1.0 | 1.0 |
| reduced | HC + LC | 97.9 | 98.0 | 97.8 | 98.0 | 98.2 | 98.1 | 98.0 | 98.0 |
| (%) | LMW | 1.0 | 1.0 | 1.0 | 1.1 | 0.9 | 1.0 | 1.0 | 1.0 |
| retained- | | 100 | 100 | 100 | 100 | 100 | 100 | 100 | 100 |
| binding | | | | | | | | | |
| Activity | | | | | | | | | |
| (%) | | | | | | | | | |

N/A corresponds to not analyzed
* measurements performed using surface plasmon resonance (SPR)
#mAb1 is included at 10 mg/ml instead of 50 mg/ml as indicated for formulations F5, F6, F7, and F8 in table 2

In the above tables, HMW refers to high molecular weight particles, LMW refers to low molecular weight particles and Monomer refers to the intact antibody as such.

Measurements have been performed using CGE, that is capillary gel electrophoresis, UP-SEC, ultra high performance size exclusion chromatography, as well asSPR, that is surface plasmon resonance.

The results show that mAb1 is readily nebulizable in both F5 as well as F7 while the antibody mAb1 is stable within these formulations.

### Example 1.6

Applicability of mAb1 in F5 via Nasal and Oral Inhalation

In order to show applicability of the formulation for nasal as well as oral inhalation measurements using artificial respiratory models have been performed.

For assessment of the complete delivery profile of the product for use in clinical trials, the ASDR/DSDR and TASD/TDSD (i.e. total dose delivered to the patient) were determined according to the procedure described in Ph. Eur. 2.9.44 and in USP <1601> with the exception that individual experiments were conducted for the measurement of these two parameters. ASDR/DSDR and TASD/TDSD were investigated using a filter-based method including an artificial lung as breathing simulator (ASL 5000, Ingmar Medical, Pittsburgh, PA, USA; 500 mL inhaled volume, tidal breathing, 15 breaths per minute, sinusoidal, 1:1 inhalation/exhalation ratio).

ASDR/DSDR was assessed to investigate the amount of mAb1 that is delivered to the patient within one minute. All measurements were performed using mouthpiece adapters.

Table 7 shows the results of three individual experiments and their mean per tested nebulizer for mAb1 formulated in F5 at 50 mg/mL. For nebulization characteriaztion with the Aerogen® Solo, a 10 mg/mL mAb1 formulation (corresponds to F3) was used in addition.

TABLE 7
ASDR/DSDR of mAb1 at 50 mg/mL and 10 mg/mL

| | | | ASDR/DSDR [mg/min] ^{a} | | | |
|---|---|---|---|---|---|---|
| | mAb1 | Nebulization | Test | Test | Test | |
| Nebulizer | conc. | time [s] | 1 | 2 | 3 | Mean |
| Aerogen ® | 10 mg/ml | 300 | 2.31 | 2.16 | 2.41 | 2.3 |
| Solo | 50 mg/mL | 60 | 8.46 | 6.71 | 7.52 | 7.6 |
| InnoSpire ® | 50 mg/mL | 60 | 9.01 | 10.3 | 8.80 | 9.4 |
| Go | | | | | | |
| AeroEclipse II | 50 mg/mL | 60 | 7.40 | 7.27 | 7.05 | 7.1 |

^{a} results are rounded to three significant digits

To collect sufficient active substance for analytical testing, the nebulization time (Aero-gen® Solo) of the 10 mg/mL solution was prolonged to 300 seconds. The delivery rate of active substance per minute increased with increasing mAb1 concentration. F5 and F3 showed overall good delivery rates independent of the nebulizer used.

In order to assess the total amount of mAb1 that is inhaled by the patient and to estimate aerosol losses within the nebulization system itself and during exhalation, TASD/TDSD was determined. Measurements were performed with the Aerogen® Solo nebulizer configuration using the mouthpiece adapter or using the face mask delivered together with the nebulizer.

The amount of total mAb1 depositing on the inhalation filter and in the mouth or the individual compartments of the nasal cast was analyzed. TASD/TDSD individual test values and their mean for mAb1 are represented in Table 8 for the mask and oral breathing and in Table 9 for the mask and nasal breathing.

TABLE 8
TASD/TDSD of mAb1 at 50 mg/mL using a face mask in combination
with an oral model (n = 3); oral breathing

| | | mAb1 API [mg] | | | | % of metered dose ^{b} | | | |
|---|---|---|---|---|---|---|---|---|---|
| Nebulizer | Compartment | Test 1 | Test 2 | Test 3 | Mean | Test 1 | Test 2 | Test 3 | Mean |
| Aerogen ® | Mouth | 1.5 | 4.9 | 2.0 | 2.8 | 0.6 | 2.0 | 0.8 | 1.1 |
| Solo ^{a} | Lung (filter) | 128.4 | 133.0 | 97.3 | 119.6 | 51.4 | 53.2 | 38.9 | 47.8 |
| | Total = | 129.9 | 137.9 | 99.3 | 122.4 | 51.9 | 55.2 | 39.7 | 48.9 |
| | TASD/TDSD | | | | | | | | |

^{a} nebulization times for Test 1, Test 2 and Test 3 were 18:52, 18:10 and 18:06 minutes, respectively.
^{b} dose of mAb1 filled into the nebulizer

TABLE 9
TASD/TDSD of mAb1 at 50 mg/mL using a mask in combination
with a nasal cast model (n = 3); nasal breathing

| | | mAb1 API [mg] | | | | % of metered dose ^{b} | | | |
|---|---|---|---|---|---|---|---|---|---|
| Nebulizer | Compartment | Test 1 | Test 2 | Test 3 | Mean | Test 1 | Test 2 | Test 3 | Mean |
| Aerogen ® | Nose | 12.8 | 11.1 | 9.5 | 11.1 | 5.1 | 4.4 | 3.8 | 4.5 |
| Solo ^{a} | Throat/trachea | 6.4 | 7.3 | 5.9 | 6.5 | 2.5 | 2.9 | 2.4 | 2.6 |
| | Subtotal | 19.2 | 18.4 | 15.4 | 17.6 | 7.7 | 7.4 | 6.1 | 7.1 |
| | Lung (filter) | 74.0 | 73.3 | 67.0 | 71.4 | 29.6 | 29.3 | 26.8 | 28.6 |
| | Total = | 93.2 | 91.7 | 82.4 | 89.1 | 37.3 | 36.7 | 32.9 | 35.6 |
| | TASD/TDSD | | | | | | | | |

^{a} nebulization times for Test 1, Test 2 and Test 3 were 16:12, 15:40 and 16:47 minutes, respectively.
^{b} dose of mAb1 filled into the nebulizer

TDSD (Total Drug Substance Delivered) is the overall amount of formulation which is deposited in the lung during the inhalation.

% metered dose is the amount of drug which is recovered during the nebulization experiments in comparison to the dosage used, i.e. the dose of mAb1 filled into the nebulizer.

Tests were performed for the Aerogen® Solo with the Ultra chamber and face mask. For the set-up with face mask and nasal breathing (table 9), a lower mean TASD/TDSD of 89.1 mg was obtained. This TASD/TDSD is the total of mean mAb1 amounts of 11.1 mg in the nasal cavity, 6.5 mg in the throat/trachea and 71.4 mg on the filter that represents the lung in this applied model.

The reduced TASD/TDSD with nasal breathing is expected since only partial deposition (i.e. deposition of particles with larger MMAD) can be expected for the aerosol fraction that enters the nasal cast but does not reach the filter at the end of the cast. The portion of non-deposited particles will then be lost via the mask outlet valves in the expiration phase.

From the TASD/TDSD for oral breathing of 119.6 mg (lung filter) and 122.4 mg (mask), a lung dose can be estimated by multiplying this with the mean FPF<5.0 μm of approximately 60% (see Table 3).

These data show that the present formulation is applicable for oral as well as nasal inhalation.

The pharmaceutical composition or the formulation as described could be demonstrated to efficiently stabilize all antibodies for inhalative administration using (i) different nebulizer systems (e.g. mesh nebulizer, jet nebulizer), (ii) diluted and undiluted formulation (different API concentration), and (iii) different masks (oral and nasal).

### Example 1.7

Assessment of the Antiviral Efficacy of Two Prophylactic Nebulizations of the mAb1 in Cynomolgus Monkeys

The antiviral efficacy of two prophylactic nebulizations of the antibody mAb1 was assessed in 6 cynomolgus monkeys prior to their infection with SARS-COV-2. For the study, 6 cynomolgus monkeys (Macaca fascicularis) were divided in two treatment groups: 4 animals were included in the treatment group that received antibody before infection, while two animals received vehicle only before infection.

Animals of the treatment group received two applications of 10 mL mAb1 (50 mg/mL in 20 mM acetate, 220 mM glycine, 20 mM trehalose, 0,04% (w/v) Polysorbate 20, pH 5,5) 4 (D-4) and 2 (D-2) days before infection. Application was with a mesh nebulizer, Aerogen Solo® nebulizer (Aerogen GmbH, Ratingen, Germany), and a suitable face mask (Laerdal Medical GmbH, Puchheim, Germany, size S).

At day of infection (D0), all animals were inoculated with 10⁷ TCID₅₀ SARS-COV-2 strain hCoV-19/France/OCC-NRC02765/2020 (accession GISAID ″EPI_ISL_640002, spike substitution D614G, K1073N) by intranasal (IN) application of 500 μL per nostril with a microsprayer device (model IA-1B, PennCenturyR®) connected to a 1 mL safety syringe with Luer Lock, and by intra-tracheal (IT) infection by spraying 1 mL of the inoculum in the trachea using a microsprayer device (model IA-1B, PennCentury™) connected to a 1 mL safety syringe with Luer Lock.

Daily blood and saliva samples, nasopharyngeal and oropharyngeal swabs were collected for analysis. Bronchoaveolar lavages were taken at D2, D4, and D6. Clinical monitoring included body temperature, food consumption, and body weight. Necropsy at D6 included histopathology of the lungs and viral load assay of lungs, nasal mucosa, oropharynx, and kidneys.

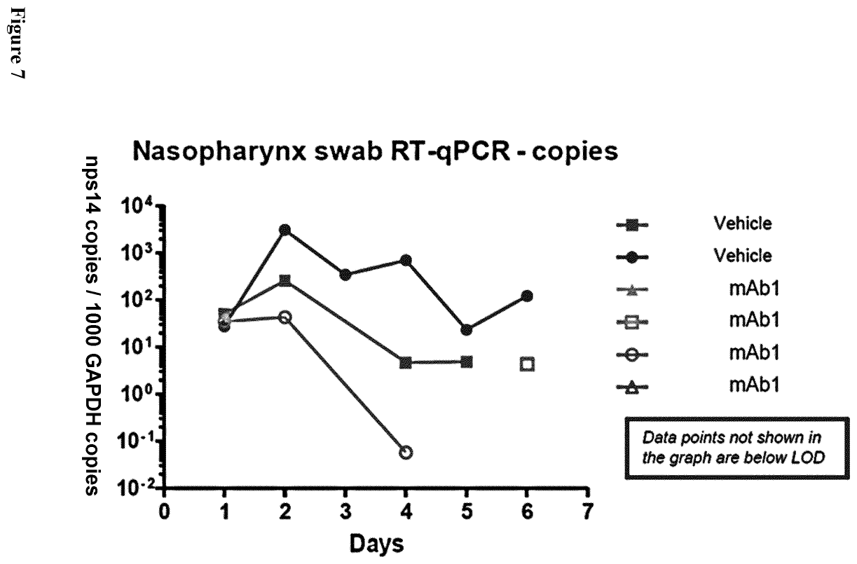

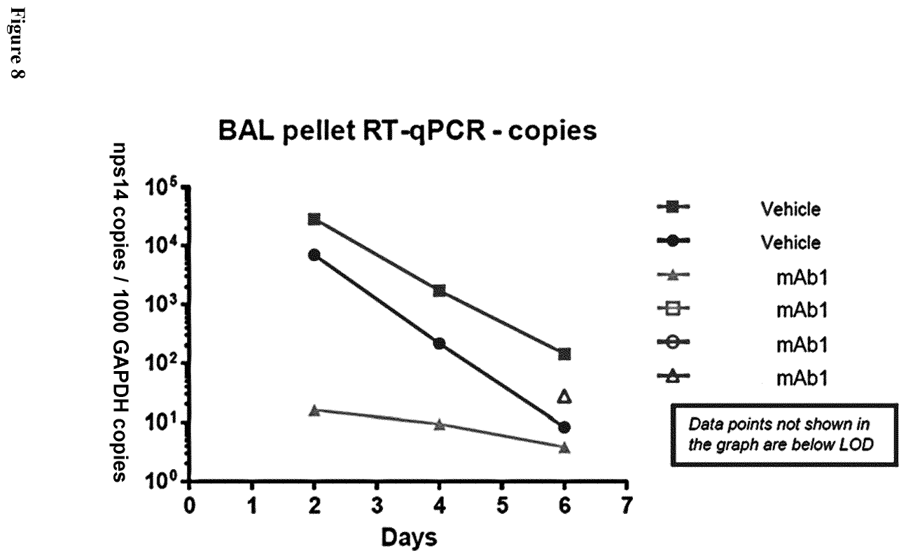

In nasopharyngeal swabs and bronchoalveolar lavage (BAL), viral copies could be found in both control animals after infection. By contrast, the treated animals showed either results below the limit of detection (LOD) or at levels several logs below that of control animals. Results of the analysis of nasopharyngeal swabs of all animals are shown in FIG. 7, while results of the analysis of bronchoalveolar lavages are shown in FIG. 8.

Body temperature: Both control animals showed a clear and prolonged hyperthermia following infection with SARS-COV-2. Prophylactic treatment with mAb1 either delayed the occurrence of hyperthermia, shortened its duration and decreased its intensity, or altogether prevented its onset.

Macroscopic and microscopic observation of lungs: Both control animal lungs showed hardened, dark red areas, similar to what has been described as lung consolidations in major publications. The lungs of the four treated animals appeared healthy without any signs of lungs injury. All these observations were confirmed by the microscopic observations: marked, extensive, subacute, bronchointerstitial inflammation in most slides of both control animals, and very limited or no bronchointerstitial inflammation in group 2 animals.

In summary, these results indicate that prophylactic treatment with mAb1 by inhalative application decreased the viral load in terms of viral copies and infectious virus, reduced or prevented clinical symptoms (hyperthermia) and prevented lung pathology.

In the following examples different formulations with mAb1, mAb2, mAb3, mAb4, and mAb5 are investigated.

### Example 2.1

### Stability of Different Antibodies in Different Formulations

The formulations according to the invention show a superior stability in comparison to other investigated formulations.

In the following, the formulations shown in table 2 have been used with different antibodies.

The stability of mAb1, mAb2, mAb3, mAb4, and mAb5 has been tested in formulation F5 (see table 2). This formulation according to the invention shows very good stability for different antibodies, both at 5° C. and at 25° C. 5° C. corresponds to normal storage conditions (intended storage conditions), which are to be expected for biomolecules. 25° C. represent accelerated storage conditions which are expected to take place only over a short period of time, e.g. prior to administration of the formulation to a subject/patient.

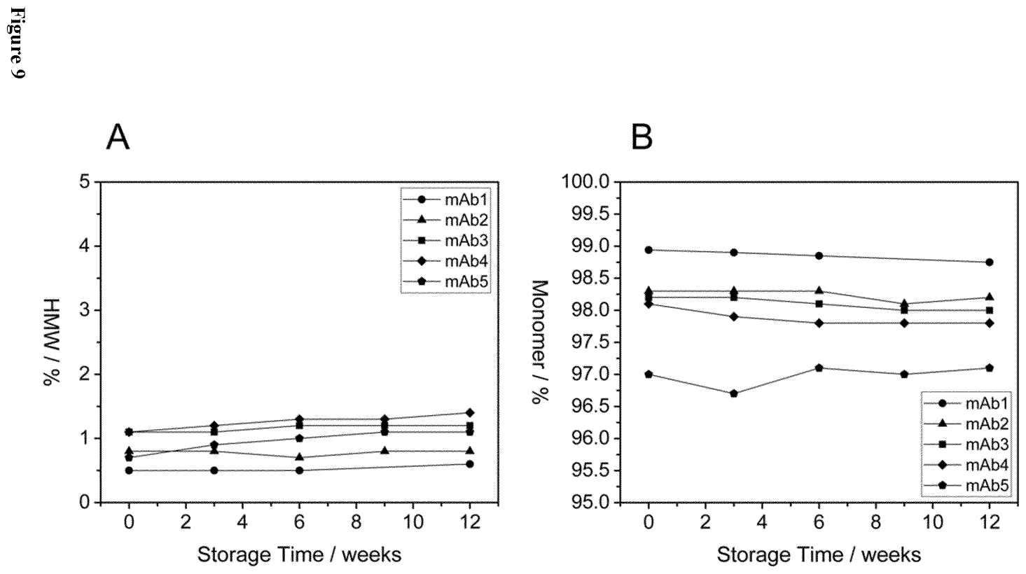

Results of these tests are shown in FIGS. 9 (5° C.) and 10 (25° C.), wherein the percentage of high molecular weight species (HMW (%)) is shown in A and the percentage of monomer (monomer (%)) is shown in B.

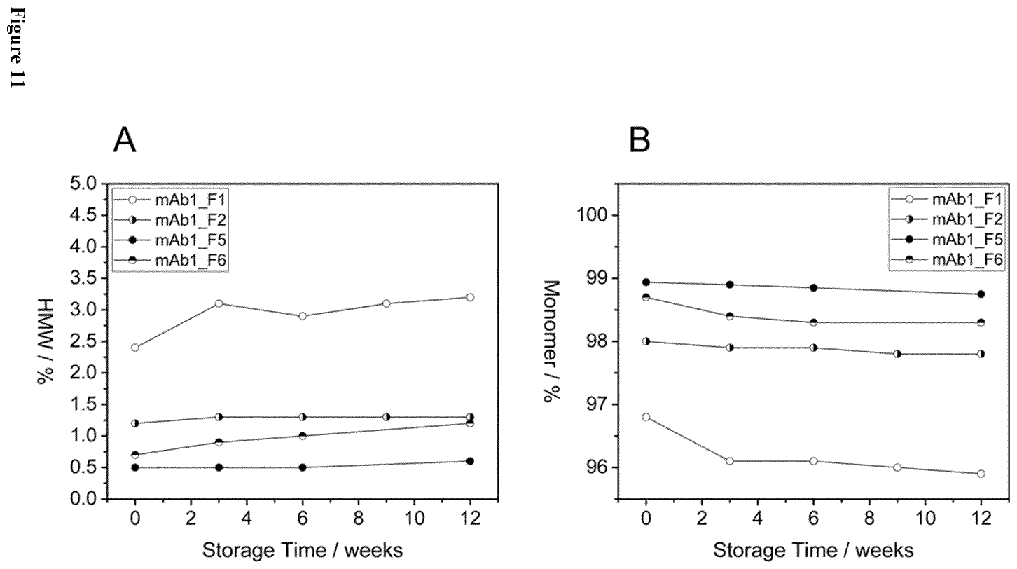

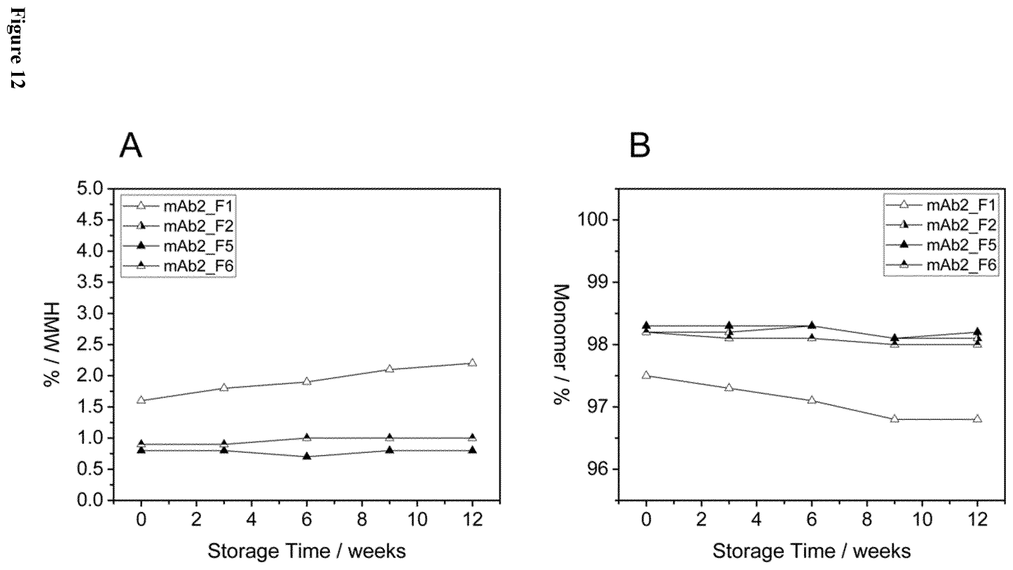

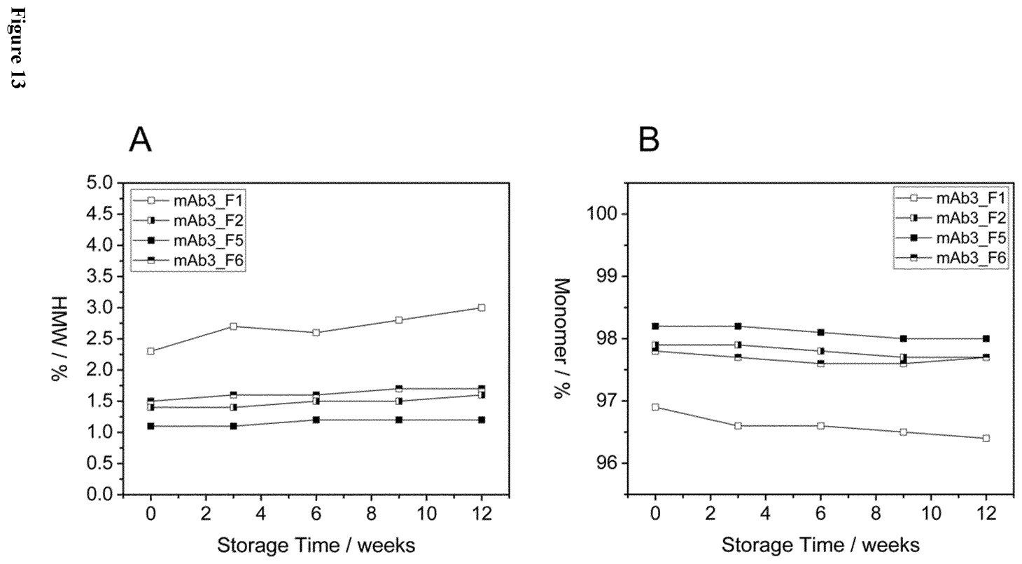

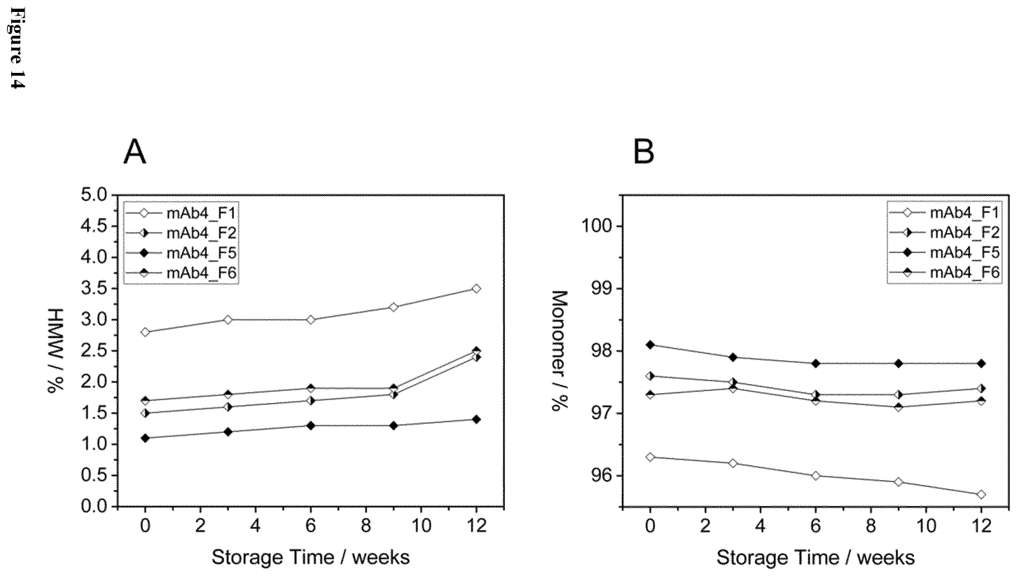

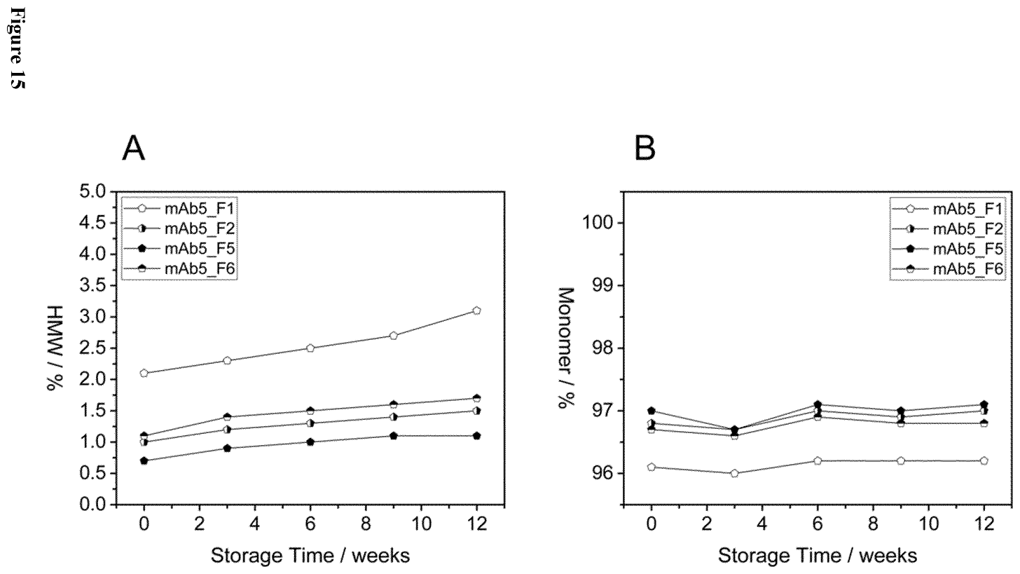

In FIGS. 11 to 15 storage stability of mAb1 (FIG. 11), mAb2 (FIG. 12), mAb3 (FIG. 13), mAb4 (FIG. 14), and mAb5 (FIG. 15) at 5° C. are shown, each in formulation F5 as well as in formulations F1, F2, and F6 which are not according to the invention. The formulations are described in table 2. In each figure the percentage of high molecular weight species (HMW (%)) is shown in A and the percentage of monomer (monomer (%)) is shown in B.

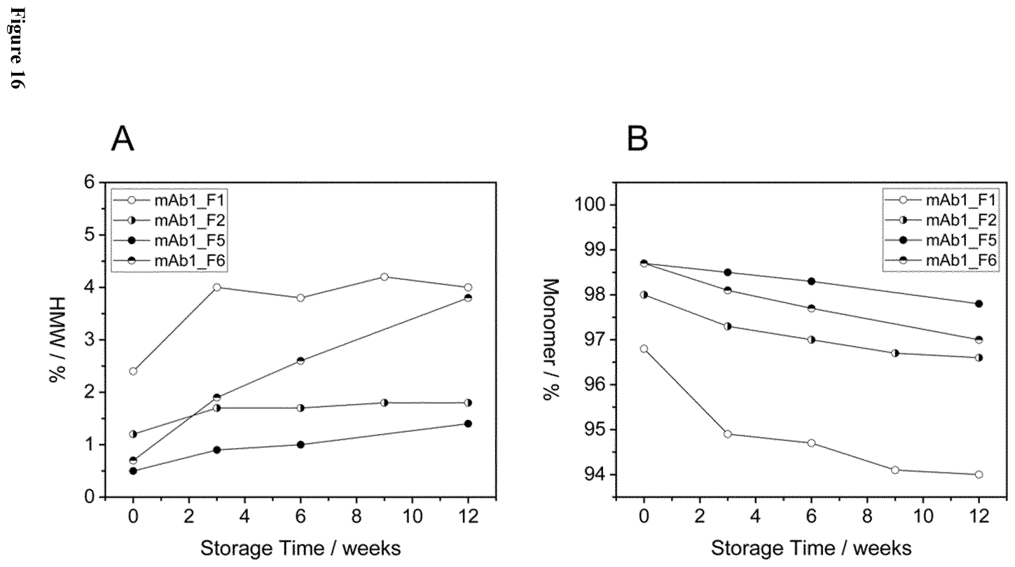

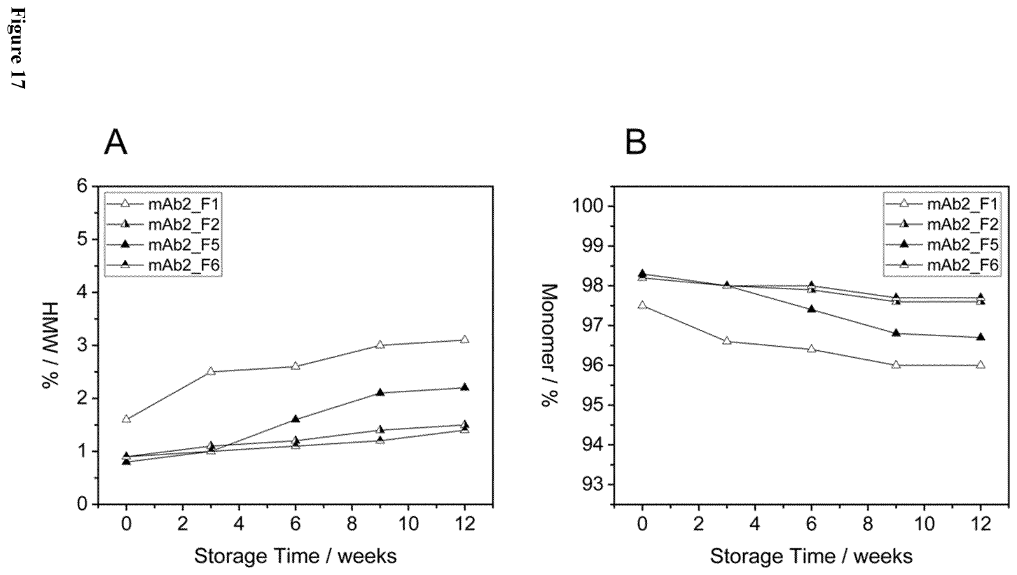

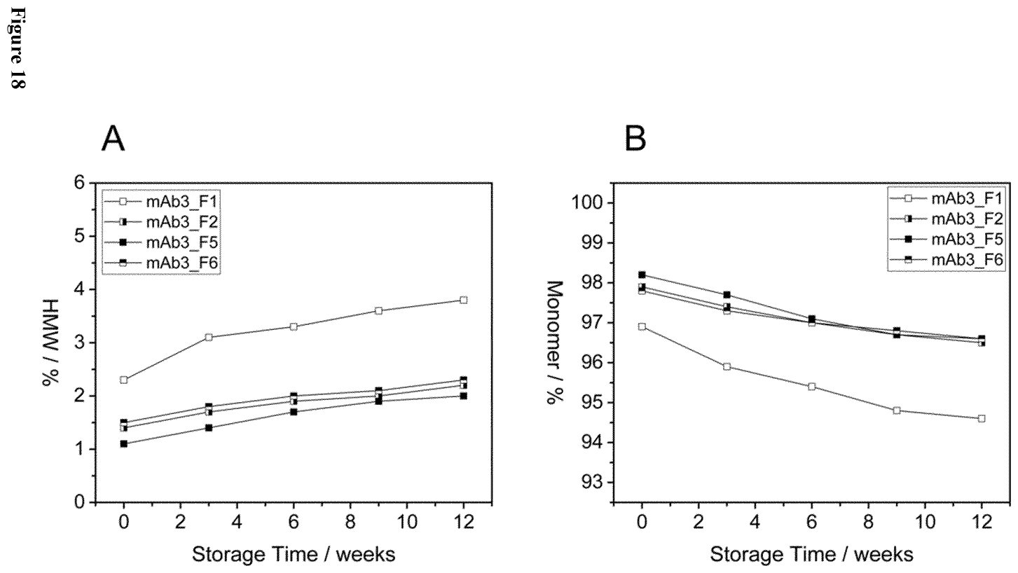

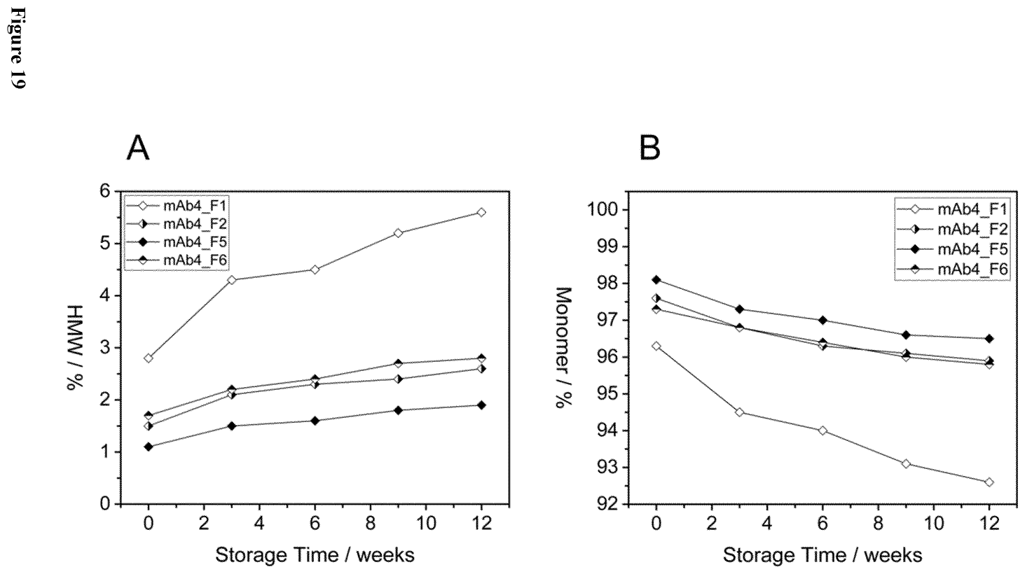

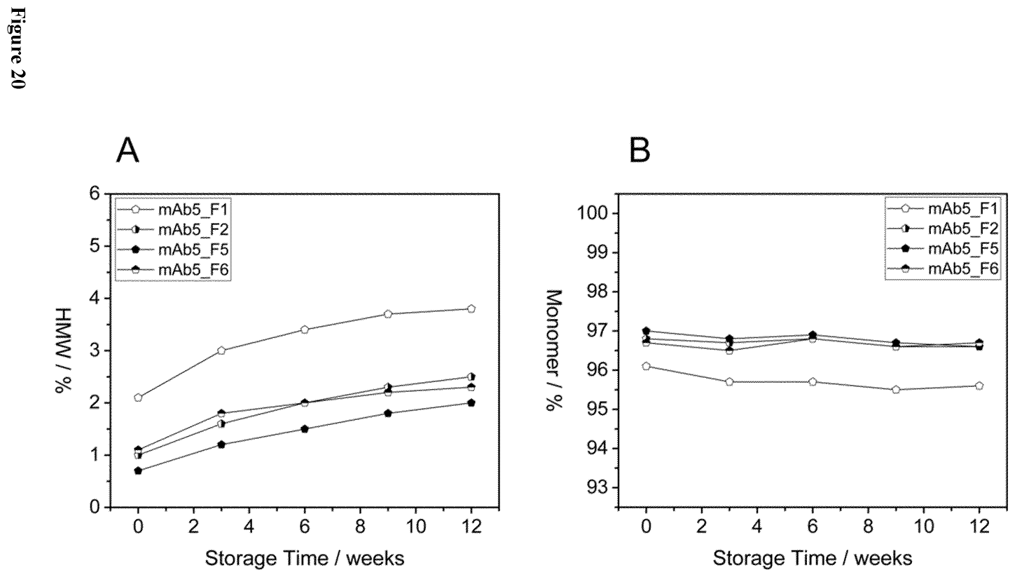

FIGS. 16 to 20 show storage stability of mAb1 (FIG. 16), mAb2 (FIG. 17), mAb3 (FIG. 18), mAb4 (FIG. 19), and mAb5 (FIG. 20) at 25° C., each in formulations F5, F1, F2, and F6 (see table 2). The percentage of high molecular weight species (HMW (%)) is shown in A and the percentage of monomer (monomer (%)) is shown in B.

High stability of all investigated antibodies in F5 could be demonstrated.

### Example 2.2

### Stability of Different Antibodies at Different Concentrations

As the pharmaceutical formulation according to the invention is intended to be used over a wide range of concentrations of an antibody or antigen-binding fragment thereof, stability of mAb1, mAb2, and mAb3 has been determined at concentrations of 10 mg/mL, 50 mg/mL and 150 mg/mL and at storage conditions of 5° C. and 25° C., respectively.

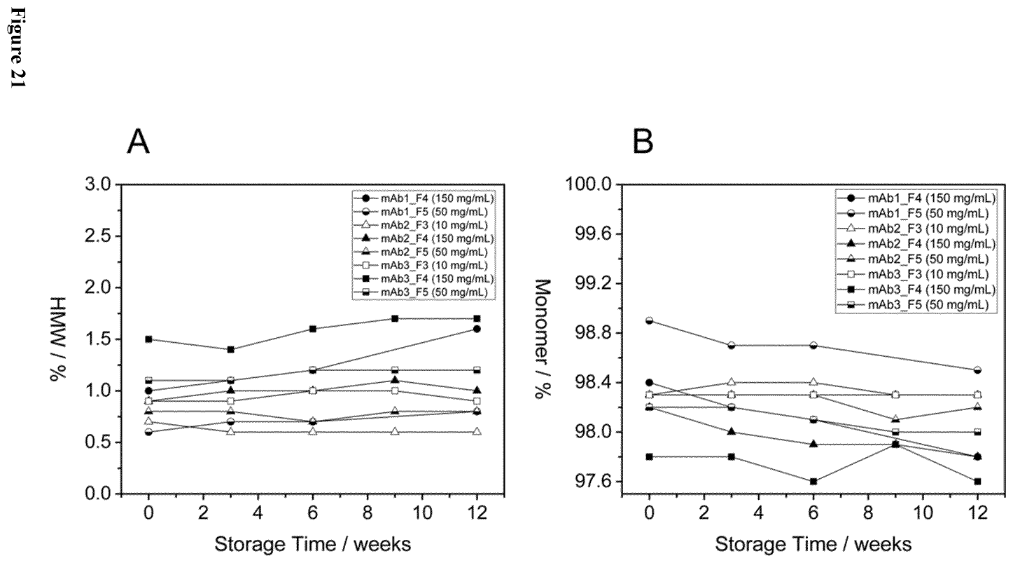

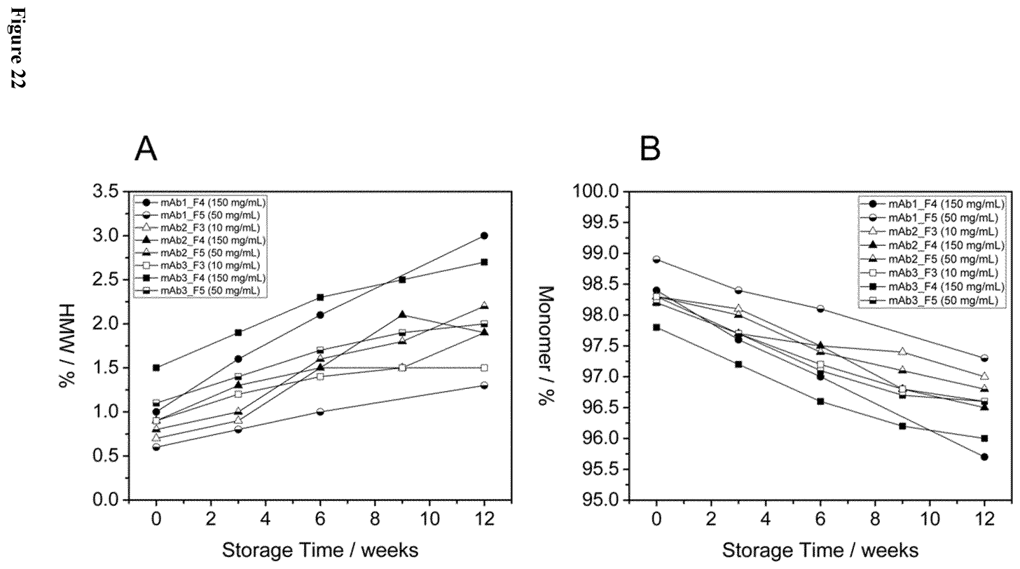

Results are shown in FIGS. 21 and 22, wherein data points are depicted for mAb1, mAb2, and mAb3 as circles (○), triangles (Δ), and squares (□), respectively. mAb2 and mAb3 concentrations were evaluated: 10, 50 and 150 mg/mL, indicated as open, half-filled, and filled forms. For mAb1 only concentrations of 50 mg/ml and 150 mg/ml were measured. The percentage of high molecular weight species (HMW (%)) is shown in A and the percentage of monomer (monomer (%)) is shown in B.

### Example 2.3

### Nebulization of Different Antibodies

Nebulization of antibodies mAb1, mAb2, mAb3, mAb4, and mAb5 has been performed using 5 mL sample volume of formulation F5 of each of these antibodies, i.e. at a concentration of the antibodies of 50 mg/mL. For the nebulization a Aerogen Solo® inhaler was used.

Aerodynamic characterization and product quality assessment was performed during and after the nebulization. All test have been done in triplicate. The mean values of the three independent tests are listed in Table 10 below.

TABLE 10
Nebulization of mAb1 to mAb5 at 50 mg/mL API concentration
in F5 using the Aerogen Solo ®

| Analytical Parameter | unit | mAb1 | mAb2 | mAb3 | mAb4 | mAb5 |
|---|---|---|---|---|---|---|
| MMAD | μm | 4.4 | 4.4 | 4.4 | 4.3 | 4.4 |
| GSD | — | 1.8 | 1.8 | 1.8 | 1.8 | 1.8 |
| FPF <5.0 μm | % | 57 | 58 | 59 | 60 | 59 |
| Nebulization time* | sec | 79 | 90 | 72 | 73 | 83 |
| (0.5 ml sample volume) | | | | | | |
| Cumulative | min | 13.2 | 15.0 | 12.0 | 12.2 | 13.8 |
| nebulization time* | | | | | | |
| (5.0 mL sample volume) | | | | | | |
| UV-Vis | mg/mL | 48.6 | 49.9 | 49.8 | 48.4 | 50.1 |
| (recovery after | | | | | | |
| nebulization) | | | | | | |
| UP-SEC | HMW % | −0.1 | +0.7 | +0.1 | +0.5 | +0.5 |
| (changes from | Monomer % | +0.1 | −0.8 | −0.1 | −0.5 | −0.5 |
| unnebulized to | LMW % | 0.0 | 0.0 | 0.0 | +0.1 | 0.0 |
| nebulized samples) | | | | | | |
| SPR | % | 100% | 100% | 100% | 100% | 100% |
| (retained binding | | | | | | |
| activity) | | | | | | |

*0.5 mL sample volume of each mAb for each individual measurement were technically nebulized (25 mg dose). The cumulative nebulization time is extrapolated considering the maximum sample volume (5 mL) that can be nebulized with the Aerogen Solo ® mesh nebulizer and would correspond to a maximum inhaled dose of 250 mg.

The Mass Median Aerodynamic Diameter (MMAD) aerodynamic diameter of a droplet or particle is equal to the diameter of a sphere with a density of 1 g/mL which has the same settling velocity. Besides the geometric diameter, the density and shape of the particle influences the aerodynamic properties. In general, MMAD values between 1 to 5 μm are considered optimal for lung deposition. Smaller particles are exhaled, while larger particles are deposited in mouth, nose, or trachea.

The Geometric Standard Deviation (GSD) is a measure of dispersion for the deviation from the mean value of the size distribution, the MMAD. It is a dimensionless number. Small values indicate a narrow size distribution, which is preferred for pulmonary application, as more droplets or particles should be in the desired range between 1 to 5 μm, if the MMAD is in this region.

The Fine Particle Fraction (FPF) is the proportion of particles or droplets in the aerosol with a diameter below 5.0 μm. This is the portion that reaches the deeper lungs after inhalation.

Ultra-performance size-exclusion chromatography (UP-SEC) was used to determine high molecular weight (HMW) and monomer content prior and after nebulization in order to evaluate the antibody integrity.

Surface plasmon resonance (SPR) was used to assess the binding activity, and thus for evaluation whether the antibodies retained function after nebulization.

In general, UV-Vis, UP-SEC, and SPR were used to assess the antibody's integrity, function, and product quality prior and after nebulization using the Aerogen Solo® mesh nebulizer.

mAb1 to mAb5 were nebulized using the Aerogen Solo® mesh nebulizer. All mAbs showed very good nebulization performance when nebulized in F5. Table 10 shows that mAb1 to mAb 5 can be nebulized in F5 while FPF<5.0 μm reflects that aerosol droplets of all mAbs enter the deep lung by simultaneous retaining its integrity, function and overall product quality. However, differences were observed with respect to nebulization time and aggregate formation (increase in HMW) and monomer content (decrease in monomer) after nebulization throughout mAb1 to mAb5.

1. A formulation comprising an antibody or antigen-binding fragment thereof in an aqueous solution at a concentration of 1-200 mg/mL, 5-50 mM acetate or histidine, 120-260 mM glycine, 15-120 mM trehalose, and 0.1-1.0 g/L polysorbate 20 (polyoxyethylene (20)-sorbitan-monolaurate), at a pH of 4.5-6.5.

2. A formulation comprising an antibody or antigen-binding fragment thereof in an aqueous solution at a concentration of 10-260 mg/mL, 10-25 mM acetate or histidine, 172.7-259.1mM glycine, 17.3-25.9 mM trehalose, and 0.2-0.6 g/L polysorbate 20 (polyoxyethylene (20)-sorbitan-monolaurate), at a pH of 5.2-5.8.

3. The formulation according to claim 1 comprising an antibody or antigen-binding fragment thereof in an aqueous solution at a concentration of 10 to 150 mg/mL, 20 mM acetate or histidine, 220 mM glycine, 20 mM trehalose, and 0.4 g/L polysorbate 20, at a pH of 5.5.

4. The formulation according to claim 1 comprising an antibody or antigen-binding fragment thereof in an aqueous solution at a concentration of 50 mg/mL in 20 mM acetate, 220 mM glycine, 20 mM trehalose, and 0.4 g/L polysorbate 20, at a pH of 5.5.

5. The formulation according to claim 1, wherein the formulation comprises two or more antibodies or antigen-binding fragment thereof.

6. The method according to claim 11, wherein the method comprises administering dosages of 10 to 50 mg/kg body weight of antibody or antigen-binding fragment thereof to the patient.

7. The method according to claim 12, wherein the formulation is administered by one or more of the following routes: intravenous, intradermal, subcutaneous, oral inhalation, nasal inhalation, mask inhalation, transdermal, transmucosal, and rectal.

8. The method according to claim 13, wherein the two different routes are selected from intravenous, oral inhalation, nasal inhalation, mask inhalation, and subcutaneous.

9-10. (canceled)

11. A method for treatment of a patient, comprising administering to the patient a formulation according to claim 1 at a dosage of the antibody or antigen-binding fragment thereof of 1 to 50 mg/kg body weight of the patient.

12. A method for treatment of a patient according to claim 11, wherein an antibody or antigen-binding fragment thereof within the formulation is administered to the patient by one or more of the following routes: injection, inhalative, and topical.

13. The method for treatment of a patient according to claim 12, wherein the administration is by at least two different routes, the two different routes being selected from intravenous, inhalative, and subcutaneous.

14. The method for treatment of a patient according to claim 11, wherein an antibody or antigen-binding fragment thereof within the formulation is administered to the patient by at least two different routes and the concentration of the antibody or antigen-binding fragment thereof within the formulation that is administered by a first of the at least two different routes contains at least twice the concentration of the antibody or antigen-binding fragment thereof compared to the concentration of the antibody or antigen-binding fragment thereof within the formulation that is administered by the second of the at least two different routes.

15. The method for treatment of a patient according to claim 11, wherein the formulation is administered to the patient by inhalation and the concentration of the antibody or antigen-binding fragment thereof in the formulation is from 10 to 150 mg/mL.

16. The method for treatment of a patient according to claim 15, wherein the concentration of the antibody or antigen-binding fragment thereof in the formulation is from 10 to 80 mg/mL.

17. The method for treatment of a patient according to claim 16, wherein the concentration of the antibody or antigen-binding fragment thereof in the formulation is from 10 to 60 mg/mL.

18. The method for treatment of a patient according to claim 17, wherein the concentration of the antibody or antigen-binding fragment thereof in the formulation is from 10 to 50 mg/mL.

19. The method according to claim 6, wherein the method comprises administering dosages of 30 to 50 mg/kg body weight of antibody or antigen-binding fragment thereof to the patient.
